(19) United States
(12) Patent Application Publication
(10) Pub. No.: US 2010/0047179 A1
(43) Pub. Date: Feb. 25, 2010

(54) TARGETED SPLIT BIOMOLECULAR CONJUGATES FOR THE TREATMENT OF DISEASES, MALIGNANCIES AND DISORDERS, AND METHODS OF THEIR PRODUCTION

(75) Inventors: Vadim Demidov, Boston, MA (US); Natalia Broude, Natick, MA (US); Charles Cantor, Del Mar, CA (US); William Evans, Memphis, TN (US)

Correspondence Address: RONALD I. EISENSTEIN, 100 SUMMER STREET, NIXON PEABODY LLP, BOSTON, MA, 02110, US

(73) Assignees: TRUSTEES OF BOSTON UNIVERSITY, Boston, MA (US); ST. JUDE CHILDREN'S RESEARCH HOSPITAL, Memphis, TN (US)

(21) Appl. No.: 12/447,368

(22) PCT Filed: Oct. 26, 2007
(86) PCT No.: PCT/US2007/082665

§ 371 Date: Jul. 23, 2009

Related U.S. Application Data
(60) Provisional application No. 60/854,892, filed on Oct. 27, 2006.

(51) Int. Cl. A61K 48/00; C07K 2/00; A61K 38/02; A61K 39/00; A61K 39/02; A61K 38/54; A61P 25/00; A61P 25/28; A61P 29/00; A61K 9/127; A61K 9/14; A61K 31/7088; A61K 49/00

(52) U.S. Cl. 424/9.6; 530/322; 514/8; 424/184.1; 424/236.1; 424/94.3; 424/450; 424/489; 514/44 R

(57) ABSTRACT

The present invention is directed to compositions and methods for the production of split-biomolecular conjugates for the directed targeting of nucleic acids and polypeptides. More preferably, the compositions and methods allow for the use of the split biomolecular conjugates for the treatment of diseases, malignancies, disorders and screening. In some embodiments, the split biomolecular conjugates comprise split effector protein fragments conjugated to a probe, and interaction of both probes with a target nucleic acid or target polypeptide, such as a pathogenic nucleic acid sequence or pathogenic protein, brings a the split-effector fragments together to facilitate the reassembly of the effector molecule. Depending on the effector molecule, the protein complementation results in a cellular effect, in particular for the treatment of diseases, malignancies and disorders.

### CROSS REFERENCE TO RELATED APPLICATIONS

This application is a 371 National Phase Entry Application of co-pending International Application PCT/US2007/082665 filed Oct. 26, 2007, which designated the U.S., and claims the benefit under 35 U.S.C. 119(e) of U.S. Provisional Application Ser. No. 60/854,892 filed on Oct. 27, 2006, the contents of which are incorporated herein by reference in their entirety.

### FIELD

The present invention is directed to compositions and methods for the production of split-biomolecular conjugates for the directed targeting of nucleic acids and polypeptides. More preferably, the compositions and methods allow for the use of the split biomolecular conjugates for the treatment of diseases, malignancies and disorders.

### BACKGROUND

Many types of tumors/cancers (including leukemias, lymphomas, sarcomas, adenomas), viral disease (including HIV/AIDS, avian flu, SARS) and other disorders are due to the cells carrying a disease specific RNA, which codes for the pathology-causing protein or pathogenic protein. Several therapeutic approaches were developed for sequence specific silencing or degradation of pathogenic RNAs by antisense oligonucleosides, ribosomes and small interfering RNAs (siRNAs). However, even when using such technologies and approaches in the treatment of pathological cells, the cells remain alive, which may result in the return or regression of the disease.

An alternative strategy is to use immunotoxins, which deliver a protein toxin preferentially to pathological cells thus selectively killing them. However, both high toxicity and high immunogenecity have limited the clinical use of immunotoxins. In these situations, high toxicity is due to the use of an entire toxin linked to the delivering antibody, hence the toxin may also target, though less efficiently, healthy cells as well. High immunogenicity is due to the regeneration of antibodies against the toxin, which circulates unprotected in the bloodstream before it is delivered to target cells by the delivering antibody.

Immunotoxins are typically composed of a targeting moiety, such as a ligand, growth factor or antibody that has cell type selectivity linked to a protein toxin or an antibody with extraordinary potency (Hall et al, 2001; Cancer Res; 81; 93-124). The targeting moiety recognizes and delivers the whole molecule to the specific receptors on the surface of the malignant cells. The toxin then triggers cell death by either (i) reaching the cytosol and catalytically inactivating vital cell process, or (ii) by modifying the tumor cell membrane. Toxins used in immunotoxins are tagged to a targeting moiety which are typically are either an antibody that recognizes and binds to a surface receptor specifically expressed on the cancer cells, or a ligand to a receptor which is specifically expressed on the surface of cancerous cells. Commonly used immunotoxins employs ribonucleases conjugated to monoclonal antibodies (MAb) (Hurset et al, 2002; 43; 953-959), often targeting the surface receptors of cancer cells and carrying toxins capable of killing the cell with a single molecule (Yamaizumi et al, 1978; 15: 245-250; Eiklid et al, 1980; 126:321-326).

There are some major limitations to the use of recombinant immunotoxins, in particular the specificity is determined by the distribution localization and expressing event of the targeting antigens. In some instances, where the target receptors are also presented in normal cells as well as tumor cells, non-specific binding and side-effects can occur.

### SUMMARY

The inventors of the present invention have discovered a method for production and use of split-biomolecular conjugates for the targeted treatment of diseases, disorders and malignancies. More specifically, the invention relates to methods to treat diseases, disorders and malignancies using a split-biomolecular conjugate comprising a split effector polypeptide, where each effector fragment is conjugated to a probe. Interaction of both probes with a target nucleic acid or target polypeptide, such as a pathogenic nucleic acid sequence or pathogenic protein, brings the effector fragments together to facilitate the reassembly, also referred to in the art as “protein complementation” of the effector molecule. Depending on the effector molecule, the protein complementation results in a cellular effect. The methods of this invention are based on therapeutic protein complementation methods.

In some embodiments, the target nucleic acid is DNA or RNA, and in some embodiments it is a nucleic acid sequence encoding a gene comprising a mutation and/or polymorphism. In other embodiments, the target nucleic acid is a nucleic acid encoding pathogenic protein, such as for example but not limited to; an oncogene, a dysfunctionally expressed protein such as inappropriately protein expression (i.e. protein expression at reduced or increased levels as compared to normal), or a protein expressed in the incorrect tissue or cell type. In other embodiments, a target nucleic acid comprises a pathogen genome or pathogen nucleic acid, for example viral (such as HIV or avian flu) or other pathogen genomes or nucleic acid sequences. In some embodiments, the target is a polypeptide, such as for example, but not limited to, a mutated protein, unfolded protein, a protein from a pathogen, an oncogene protein etc.

In some embodiments, the effector molecule component of the split-biomolecular conjugate is a toxin, for example a bacterial or plant toxin. In other embodiments, the effector molecule is a nuclease, for example a DNase or RNase. In other embodiments, the effector molecule is a cytotoxin, for example a cytokine. In other embodiments, the effector molecule is a protease molecule, and in other embodiments, the effector molecule induces a cell death pathway, for example the effector molecule can be a pro-apoptotic molecules such as Bad, bax and other pro-apoptotic proteins commonly known by persons of ordinary skill in the art. In an alternative embodiment, the effector molecule inhibits cell death or induces cell survival pathway induction, for example anti-apoptotic molecules such as members of the bcl-2 family and IAP protein family.

In another embodiment, an effector molecule is a sensitizing molecule which catalyzes a secondary agent into a cytotoxic molecule, for example but not limited to an effector molecule such as HGPRT which catalyzes the prodrug allopurinol into a molecule that has a cytotoxic function. Other examples of effector molecules useful in the methods as disclosed herein include molecules that modify the target nucleic acid or target polypeptide, for instance DNA methyltransferases and ubiquitination E3 enzymes to silence gene expression or induce protein degradation respectively.

In some embodiments, the probe component of the split-biomolecular conjugate is a nucleic acid, for example DNA, RNA, PNA, pcPNA etc, and in other embodiments, the probe is a polypeptide. Both the nucleic acid and polypeptide probes are capable of binding and recognizing nucleic acid and polypeptide targets.

In some embodiments, a split-biomolecular conjugate as disclosed herein is capable of inducing cell death, and comprises an effector molecule that enables this functionality. In such embodiments, a split-biomolecular conjugate can be used in the treatment of cancers, pathogens (for example viral infections) and any other disorder or disease, for example immune disorders where targeted cell death is the desired function.

In some embodiments, a split-biomolecular conjugate useful in the methods as disclosed herein is capable of degrading the target nucleic acid or target polypeptide and comprises an effector molecule that enables this functionality, for example effector molecules such as a proteases or DNA/RNA nucleases. In such an embodiment, a split nucleic acid can be used for many therapeutic applications, for example for the treatment and/or prevention of cancers, pathogen infections, and the treatment of cells and disorders due to the expression of pathogenic nucleic acid and/or pathogenic polypeptide. In some embodiments, expression of a pathogenic nucleic acid and/or pathogenic polypeptide can occur as the result of a mutation, single nucleic acid polymorphisms (SNPs) etc. In some embodiments, the split-biomolecular conjugates as disclosed herein are useful in the treatment of disorders or disease where expression of a protein contributes to, wholly or in part, at least one symptom of the disease. In some embodiments, diseases which can be treated by the methods as disclosed herein include for example, but are not limited to, neurodegenerative disorders, immune disorders, cancers and presence of pathogenic nucleic acid/polypeptides.

In other embodiments, a split-biomolecular conjugate as disclosed herein is capable of sensitizing the cell to subsequent insult by a second agent, and comprises an effector molecule which is an enzyme or molecule capable of catalyzing a prodrug into a molecule which function as a cytotoxin. In such embodiments, sensitizing split-biomolecular conjugates are useful for selective cell death in the targeted cells. Such a device is useful particularly useful when multiple insults are required for the death of cells, for example for the treatment of drug resistant cancer and virus infected cells.

In another embodiment of the invention provides methods for the production of a pharmaceutical composition comprising the split-biomolecular conjugates. In one such embodiment, the pharmaceutical is a novel delivery method for administrating the split-biomolecular conjugates, for example via preloaded polymeric nanoparticles and/or cationic liposomes.

One aspect of the present invention relates to a split biomolecular conjugate, comprising a split-effector molecule, wherein the split-effector polypeptide fragments are conjugated to one of at least two probes specific for a target nucleic acid or target polypeptide, wherein the target nucleic acid or target polypeptide is present in a cell suffering from a disease, malignancy or disorder, wherein binding of the probes to the target nucleic acid or polypeptide reconstitutes the effector molecule, and wherein the effector molecule is; lethal to the cell; and/or sensitizes the cell to another compound; and/or alleviates the disease, malignancy or disorder.

In some embodiments, a split biomolecular conjugate can comprise a split-effector molecule comprising at least two polypeptide fragments of an effector molecule; wherein the fragments; (a) are in an activated conformation; (b) are not active by themselves; (c) further comprise a probe; and (d) complement to reconstitute the active effector molecule in real time in the presence of a target nucleic acid or polypeptide.

Another aspect of the present invention relates to a method for the treating or reducing the effects of a disease or disorder in a subject comprising administering to the subject an effective amount of a pharmaceutical composition of the split biomolecular conjugate as disclosed herein, which comprises a split-effector molecule, wherein each of the split-effector polypeptide fragments are conjugated to at least one of two probes specific for a particular target nucleic acid or target polypeptide that is associated with a disease or disorder; and formation of an active effector molecule, wherein the formation of an active effector molecule is facilitated by binding of at least two probes with the target nucleic acid or target polypeptide that is associated with a disease or disorder.

In some embodiments, a split effector molecule is a toxin molecule or fragment thereof, or alternatively, an immunotoxin or fragment thereof. In some embodiments, the split effector molecules which are toxins or immunotoxins are, but are not limited to; protein toxin, bacterial toxin and plant toxin. Examples of plant toxins useful as effector molecules in the methods as disclosed herein include, but are not limited to, plant halotoxins, class II ribosome inactivating protein, plant hemitoxins, class I ribosome inactivating protein. Further examples of plant toxins useful as effector molecules in the methods as disclosed herein include, but are not limited to, saporin (SAP); pokeweed antiviral protein (PAP); bryodin 1; bouganin and gelonin or naturally occurring variants, or genetically engineered variants or fragments thereof.

Examples of plant toxins useful in the methods as disclosed herein include, but are not limited to, anthrax toxin; diphtheria toxin (DT); pseudomonal endotoxin (PE); streptolysin 0; or naturally occurring variants, or genetically engineered variants or fragments thereof.

Further examples of plant toxins useful as effector molecules in the methods as disclosed herein include, but are not limited to, ricin A chain (RTA); ricin B (RTB); abrin; mistletoe, lectin and modeccin or naturally occurring variants, or genetically engineered variants or fragments thereof. In some embodiments, a plant toxin is a ribotoxin, for example but not limited to ricin A chain (RTA).

In some embodiments, a split effector molecule is cytotoxic molecule or fragment thereof, for example a cytokine, such as, but not limited to, IL-1; IL-2; IL-3; IL-4; IL-13; interferon-alpha; tumor necrosis factor-alpha (TNFα); IL-6; granulosa colony stimulating factor (G-CSF); GM-CSF or natural variants or genetically engineered variants thereof.

In further embodiments, a plant toxin can be a nuclease, for example but not limited to sarcin; restrictocin. In some embodiments, a split effector molecule useful in the methods as disclosed herein is a nuclease or has endonucleolytic activity, for example a DNA nuclease or DNA endonuclease, for example DNA endonuclease I or natural variants or genetically engineered variant thereof. In alternative embodiments, a nuclease can be a RNA nuclease or RNA endonuclease, for example but not limited to RNA endonuclease I; RNA endonuclease II; RNA endonuclease III. In some embodiments, a RNA nuclease can be for example, but not limited to angliogenin, Dicer, RNase A or variants or fragments thereof.

In alternative embodiments, effector molecules useful in the methods as disclosed herein include proteolytic enzymes, such as, but not limited to caspase enzymes; calpain enzymes; cathepsin enzymes; endoprotease enzymes; granzymes; matrix metalloproteases; pepsins; pronases; proteases; proteinases; rennin; trypsin or variants or fragments thereof.

Another aspect of the present invention relates to a split bimolecular conjugate comprising split effector molecules that are capable of inducing a cell death pathway in the cell. In such embodiments, effector molecules useful in the methods as disclosed herein include a pro-apoptotic molecules, such as but not limited to Hsp90; TNFα; DIABLO; BAX; inhibitors of Bcl-2; Bad; poly ADP ribose polymerase-1 (PARP-1): Second Mitochondrial-derived Activator or Caspases (SMAC); apoptosis inducing factor (AIF); Fas (also known as Apo-1 or CD95); Fas Ligand (FasL) or variants or fragments thereof.

Another aspect of the present invention relates to a split bimolecular conjugate comprising split effector molecules that are capable of inhibiting a cell death pathway or inducing a cell survival pathway in the cell. In such embodiments, effector molecules useful in the methods as disclosed herein include an anti-apoptotic molecule, for example but not limited to, bcl-2; Bcl-XL; Hsp27; inhibitors of apoptosis (IAP) proteins.

Another aspect of the present invention relates to a split bimolecular conjugate comprising split effector molecules that are capable of sensitizing a cell to one or more secondary agents. In such embodiments, effector molecules useful in the methods as disclosed herein include, but are not limited to β-gluctonidase; hypoxanthine-guianine phosphoribosynltransferase; β-lactamase; carboxylesterase HCE1; peroxidase enzyme and variants or fragments thereof. In some embodiments, a secondary agent is a antiviral drug; selected from a group comprising; oseltemivir; allopurinol.

Another aspect of the present invention relates to a split bimolecular conjugate comprising an effector molecule capable of tagging a target polypeptide for protein degradation. In such embodiments, effector molecules useful in the methods as disclosed herein include, but are not limited to, ubiquitin; Small Ubiquitin-related Modifier (SUMO); DNA methyltransferase (DNA MTase); Histone acetylation enzyme (HAT) and variants or fragments thereof.

In some embodiments, the split bimolecular conjugate as disclosed herein is useful for the treatment of a disease or disorder due to a pathology causing nucleic acid. Examples of such disease or disorders include, but by no way a limitation, cancer; neurological disease; degenerative disease; an inflammatory disease; a pathogen infection.

In some embodiments, cancer that can be treated with the split biomolecular conjugate as disclosed herein include, for example but are not limited to, mescenchymal in origin (sarcomas); fibrosarcomas; myxosarcomas; liposarcomas; chondrosarcomas; osteogenic sarcomas; angiosarcomas; endotheliosarcomas; lymphangiosarcomas; synoviosarcomas; mesotheliosarcomas; Ewing's tumors; myelogenous leukemias; monocytic leukemias; malignant leukemias; lymphocytic leukemias; plasmacytomas; leiomyosarcomas; and rhabdomyosarcoma; cancers epithelial in origin (carcinomas); squamous cell or epidermal carcinomas; basal cell carcinomas; sweat gland carcinomas; sebaceous gland carcinomas; adenocarcinomas; papillary carcinomas; papillary adenocarcinomas; cystadenocarcinomas; medullary carcinomas; undifferentiated carcinomas (simplex carcinomas); bronchogenic carcinomas; bronchial carcinomas; melanocarcinomas; renal cell carcinomas; hepatocellular carcinomas; bile duct carcinomas; transitional cell carcinomas; squamous cell carcinomas; choriocarcinomas; seminomas; embryonal carcinomas; malignant teratomas; and terato carcinomas; leukemia; acute lymphocytic leukemia and acute myelocytic leukemia (myeloblastic, promyeloblastic, myelomonocytic; monocytic, and erythroleukemia); chronic leukemia; chronic myelocytic (granulocytic) leukemia; chronic lymphocytic leukemia; polycythemia vera; lymphoma; Hodgkin's disease; non-Hodgekin's disease; multiple mycloma; Waldenstrom's macroglobulinemia; heavy chain disease. In some embodiments, the cancer is lymphia; leukemia; sarcoma; adenomas, and in some embodiments, the cancer is acute lympoblastic leukemia (ALL).

In some embodiments, neurological disease or disorders that can be treated with the split biomolecular conjugate as disclosed herein include, for example but are not limited to, Alzheimer's Disease; Parkinson's disease; Huntington's disease; polyglutamine disease; Amyotrophic lateral sclerosis (ALS).

In some embodiments, pathogens that can be treated with the split biomolecular conjugate as disclosed herein include, for example but are not limited to, influenza, virus, bacteria, fungus, parasite or yeast.

In alternative embodiments, a disease that can be treated using the split biomolecular conjugate as disclosed herein is a genetic predisposition to a disease.

In some embodiments, a subject to be administered a split biomolecular conjugate as disclosed herein is a mammal, for example a human. In some embodiments, a split biomolecular conjugate is administered to a cell, for example a cell in vivo. In alternative embodiments, a cell is obtained from the subject and administered the pharmaceutical composition ex vivo, and can be, for example, transplanted back into the subject such as a human subject.

In some embodiments, the split-biomolecular conjugate is produced by inclusion bodies, and alternative embodiments, the cell is split-biomolecular conjugate is produced by cell-free system or by a bacterial expression system that minimizes the formation of inclusion bodies.

In some embodiments, a split-biomolecular conjugate is administered to a cell by a group comprising; pump; direct injection; topical application; administration to a subject via intradermal, subcutaneous; intravenous; intralymphatic; intranodal; intramucosal or intramuscular administration. Alternatively, a split-biomolecular conjugate can be administered to a cell on preloaded polymetric nanoparticles and/or cataionic liposomes.

In some embodiments, a split-effector molecule conjugated a nucleic acid binding motif is expressed from a expression vector in said cell, where the vector is introduced into the cell by conventional means. In some embodiments, vector also comprises the effector molecule.

In some embodiments, a target nucleic acid comprises the pathology causing target nucleic acid sequence, for example but not limited to DNA, RNA such as pathogenic DNA or pathogenic RNA. In some embodiments, the pathologic DNA comprises at least one mutation and/or polymorphism. In some embodiments a target nucleic acid sequence can comprise pathogen DNA or RNA, such as pathogen DNA or RNA of viral origin. In some embodiments, examples of pathogen DNA or RNA that can be targeted by the methods as disclosed herein, include for example but is not limited to, AIDS/HIV; avian flu; SARS; Hepatitis type A; Hepatitis type B; Hepatitis Type C; influenzia; varicella; adenovirus, HSV-2; HSV-II; rinderpest rhinovirus; echnovirus; rotavirus; respiratory syncytial virus; papilloma virus; papova virus; cytomegalovirus; echinovirus; abovirus; hantavirus; coxsackie virus; measles virus; mumps virus; rubella virus; polio virus; HIV-I, HIV-II; avian and/or bird flu virus; ebola virus; other viruses.

In some embodiments, a target polypeptide targeted by the split-biomolecular conjugate comprises a pathogenic polypeptide, such as but not limited to, a pathogenic polypeptide that has an abnormal conformation relative to normal non-pathogenic polypeptide.

In some embodiments, a cell is in vitro or in vivo.

In some embodiments, the split-biomolecular conjugate as disclosed herein comprises a probe that is a nucleic acid binding motif, for example but not limited to a nucleic acid binding motif such as DNA, RNA, PNA, LNA, pcPNA, DNA-binding protein, RNA-binding protein or analogues or fragments thereof.

In alternative embodiments, the split-biomolecular conjugate as disclosed herein comprises a probe that is a polypeptide detector protein. In some embodiments, a polypeptide detector protein can be split into at least two fragments, wherein each fragment is conjugated to at two or more fragments of the effector protein, and wherein the binding of the detector polypeptide fragments to the target nucleic acid or target polypeptide reconstitutes the detector protein and the active effector protein. In further embodiments, a polypeptide detector protein is a multi-domain polypeptide detector protein, for example where each domain of the multi-domain polypeptide detector protein is conjugated to at two or more fragments of the effector protein, and wherein on binding of the domains of the detector protein to the target nucleic acid or target polypeptide reconstitutes the detector protein and the active effector protein.

Another aspect of the present invention provides a cell death split-biomolecular conjugate comprising; a split-effector molecule, wherein the split-effector polypeptide fragments comprise at least two polypeptide fragments which are each conjugated to two or more probes, wherein the split-effector polypeptide fragments combine to form an active effector molecule in the presence of a particular target nucleic acid or target polypeptide that is capable of initiating a cell death pathway in the cell.

In some embodiments, the present invention provides a methods to treat cancer with a cell death split-biomolecular conjugate, the method comprising contacting a tumor cell with the cell death split-effector molecule; wherein (i) the split-effector polypeptide fragments are conjugated to probes specific for a particular target nucleic acid or target polypeptide that is associated with the cancer disorder; and wherein (ii) the split-effector polypeptide fragments combine to form an active effector molecule in the presence of a particular target nucleic acid or target polypeptide that is associated with the tumor.

In some embodiments, the present invention provides a methods to treat a pathogen infection with a cell death split-biomolecular conjugate, the method comprising; contacting a pathogen infected cell with the cell death split-effector molecule; wherein (i) the split-effector polypeptide fragments are conjugated to probes specific for a particular target nucleic acid or target polypeptide that is associated with the pathogen infection; and wherein (ii) the split-effector polypeptide fragments combine to form an active effector molecule in the presence of a particular target nucleic acid or target polypeptide that is associated with the pathogen infection.

In some embodiments, the cell death split-biomolecular conjugate is useful to kill cells comprising a pathological nucleic acid and/or pathological polypeptide, for example target nucleic acids or target polypeptides which change in the cell as an effect of the pathology. In some embodiments, a target nucleic acid for a cell death split-biomolecular conjugate encodes an oncogene or pro-oncogene, such as, but not limited to, p63; p73; gp40 (v-fms); p21 (ras); p55 (v-myc); p65 (gag-jun); pp 60 (v-src); v-abl; v-erb; v-erba; v-fos or variants thereof. Alternatively, a cell death split-biomolecular conjugate can be used to detect a pathogenic protein or pathogenic nucleic acid, were a pathogen is, for example, but not limited to influenza, virus, bacteria, fungus, parasite or yeast.

In some embodiments, the virus is selected from a group comprising; AIDS/HIV; avian flu; SARS; Hepatitis type A; Hepatitis type B; Hepatitis Type C; influenzia; varicella; adenovirus, HSV-2; HSV-II; rinderpest rhinovirus; echnovirus; rotavirus; respiratory syncytial virus; papilloma virus; papova virus; cytomegalovirus; echinovirus; abovirus; hantavirus; coxsackie virus; measles virus; mumps virus; rubella virus; polio virus; HIV-I, HIV-II; avian and/or bird flu virus; ebola virus; other viruses.

In some embodiments, a split-biomolecular conjugate, such as cell death split-biomolecular conjugate is useful for targeting nucleic acids or target polynucletides such as, but not limited to, v-fms; v-myc; v-src; v-abl; v-erb; v-erba; v-fos; M1 protein; virus like particles (VPL).

Another aspect of the present invention, the split biomolecular conjugate is a degrading split-biomolecular conjugate comprising a split-effector molecule, wherein the split-effector polypeptide fragments comprise at least two polypeptide fragments which are each conjugated to two or more probes, wherein the split-effector polypeptide fragments combine to form an active effector molecule in the presence of a particular target nucleic acid or target polypeptide that is capable of degrading or inducing the degradation of the target nucleic acid or target polypeptide in the cell.

In some embodiments, a degrading split-biomolecular conjugate is useful for the treatment of cancer, for example, the method comprising contacting a tumor cell with the cell death split-effector molecule; wherein (i) the split-effector polypeptide fragments are conjugated to probes specific for a particular target nucleic acid or target polypeptide that is associated with the cancer disorder; and wherein (ii) the split-effector polypeptide fragments combine to form an active effector molecule in the presence of a particular target nucleic acid or target polypeptide that is associated with the tumor.

In further embodiments, a degrading split-biomolecular conjugate as disclosed herein is useful in methods for the treatment of a pathogen infection, the method comprising; contacting a pathogen infected cell with the cell death split-effector molecule; wherein (i) the split-effector polypeptide fragments are conjugated to probes specific for a particular target nucleic acid or target polypeptide that is associated with the pathogen infection; and wherein (ii) the split-effector polypeptide fragments combine to form an active effector molecule in the presence of a particular target nucleic acid or target polypeptide that is associated with the pathogen infection.

In further embodiments, a degrading split-biomolecular conjugate as disclosed herein is useful in methods for the treatment of a disease or disorder, the method comprising; contacting a pathogen infected cell with the cell death split-effector molecule; wherein (i) the split-effector polypeptide fragments are conjugated to probes specific for a particular target nucleic acid or target polypeptide that is associated with the pathogen infection; and wherein (ii) the split-effector polypeptide fragments combine to form an active effector molecule in the presence of a particular target nucleic acid or target polypeptide that is associated with the disease or disorder.

In some embodiments, a degrading split-biomolecular conjugate as disclosed herein targets the degradation of a target nucleic acid, such as pathological nucleic acid or a target polypeptide, such as a pathological polypeptide, where the target nucleic acid or target polypeptide is identified as nucleic acids or polypeptides that effect of the pathology. In some embodiments, a target nucleic acid for a degrading split-biomolecular conjugate encodes an oncogene or pro-oncogene, such as, but not limited to, p63; p73; gp40 (v-fms); p21 (ras); p55 (v-myc); p65 (gag-jun); pp 60 (v-src); v-abl; v-erb; v-erba; v-fos or variants thereof. Alternatively, a degrading split-biomolecular conjugate can be used to detect a pathogenic protein or pathogenic nucleic acid, were a pathogen is, for example, but not limited to influenza, virus, bacteria, fungus, parasite or yeast.

In some embodiments, the virus is selected from a group comprising; AIDS/HIV; avian flu; SARS; Hepatitis type A; Hepatitis type B; Hepatitis Type C; influenzia; varicella; adenovirus, HSV-2; HSV-II; rinderpest rhinovirus; echnovirus; rotavirus; respiratory syncytial virus; papilloma virus; papova virus; cytomegalovirus; echinovirus; abovirus; hantavirus; coxsackie virus; measles virus; mumps virus; rubella virus; polio virus; HIV-I, HIV-II; avian and/or bird flu virus; ebola virus; other viruses.

In some embodiments, a split-biomolecular conjugate, such as degrading split-biomolecular conjugate is useful for targeting nucleic acids or target polynucleotides such as, but not limited to, v-fms; v-myc; v-src; v-abl; v-erb; v-erba; v-fos; M1 protein; virus like particles (VPL).

In some embodiments, a split-biomolecular conjugate, such as degrading split-biomolecular conjugate is useful for the treatment of a disease or disorder associated with the expression of a pathological polypeptide, such as a mutated and/or incorrectly folded polypeptide. Such diseases include, but are not limited to, neurological diseases; kidney diseases; cardiovascular diseases; hepatic diseases; inflammatory diseases; cystic fibrosis; neurodegenerative diseases; inflammatory diseases; immune disorders and the like. In some embodiments, a degrading split-biomolecular conjugate is useful for the treatment of neurological diseases such as, but not limited to Alzheimer's Disease, Huntington's disease; Parkinson's disease; amyotrophic lateral sclerosis (ALS), spinal cord injury.

Another aspect of the present invention, the split biomolecular conjugate is a sensitizing split-biomolecular conjugate comprising a split-effector molecule, wherein the split-effector polypeptide fragments comprise at least two polypeptide fragments which are each conjugated to two or more probes, wherein the split-effector polypeptide fragments combine to form an active effector molecule in the presence of a particular target nucleic acid or target polypeptide that is capable of sensitizing the cell to other secondary agents.

In some embodiments, a sensitizing split-biomolecular conjugate as disclosed herein can be used to treat cancer, the method comprising; contacting a tumor cell with the cell death split-effector molecule; wherein (i) the split-effector polypeptide fragments are conjugated to probes specific for a particular target nucleic acid or target polypeptide that is associated with the cancer disorder; and wherein (ii) the split-effector polypeptide fragments combine to form an active effector molecule in the presence of a particular target nucleic acid or target polypeptide that is associated with the tumor.

In some embodiments, a sensitizing split-biomolecular conjugate as disclosed herein can be used to a pathogen infection, the method comprising; contacting a pathogen infected cell with the cell death split-effector molecule; wherein (i) the split-effector polypeptide fragments are conjugated to probes specific for a particular target nucleic acid or target polypeptide that is associated with the pathogen infection; and wherein (ii) the split-effector polypeptide fragments combine to form an active effector molecule in the presence of a particular target nucleic acid or target polypeptide that is associated with the pathogen infection.

In some embodiments, a sensitizing split-biomolecular conjugate as disclosed herein targets the degradation of a target nucleic acid, such as pathological nucleic acid or a target polypeptide, such as a pathological polypeptide, where the target nucleic acid or target polypeptide is identified as nucleic acids or polypeptides that effect of the pathology. In some embodiments, a target nucleic acid for a sensitizing split-biomolecular conjugate encodes an oncogene or pro-oncogene, such as, but not limited to, p63; p73; gp40 (v-fms); p21 (ras); p55 (v-myc); p65 (gag-jun); pp 60 (v-src); v-abl; v-erb; v-erba; v-fos or variants thereof. Alternatively, a sensitizing split-biomolecular conjugate can be used to detect a pathogenic protein or pathogenic nucleic acid, were a pathogen is, for example, but not limited to influenza, virus, bacteria, fungus, parasite or yeast.

In some embodiments, the virus is selected from a group comprising; AIDS/HIV; avian flu; SARS; Hepatitis type A; Hepatitis type B; Hepatitis Type C; influenzia; varicella; adenovirus, HSV-2; HSV-II; rinderpest rhinovirus; echnovirus; rotavirus; respiratory syncytial virus; papilloma virus; papova virus; cytomegalovirus; echinovirus; abovirus; hantavirus; coxsackie virus; measles virus; mumps virus; rubella virus; polio virus; HIV-I, HIV-II; avian and/or bird flu virus; ebola virus; other viruses.

In some embodiments, a split-biomolecular conjugate, such as sensitizing split-biomolecular conjugate is useful for targeting nucleic acids or target polynucleotides such as, but not limited to, v-fms; v-myc; v-src; v-abl; v-erb; v-erba; v-fos; M1 protein; virus like particles (VPL).

In some embodiments, a split-biomolecular conjugate, such as sensitizing split-biomolecular conjugate comprises an effector protein such as, but not limited to, β-gluctonidase; hypoxanthine-guianine phosphoribosynltransferase; β-lactamase; carboxylesterase HCE1; peroxidase enzyme or variants or fragments thereof.

Another aspect of the present invention, the split biomolecular conjugate is a survival split-biomolecular conjugate comprising; a split-effector molecule, wherein the split-effector polypeptide fragments comprise at least two polypeptide fragments which are each conjugated to two or more probes, wherein the split-effector polypeptide fragments combine to form an active effector molecule in the presence of a particular target nucleic acid or target polypeptide that is capable of initiating a cell survival pathway or inhibiting cell death in the cell.

In some embodiments, a survival split-biomolecular conjugate as disclosed herein can be used to treat a disease or disorder, the method comprising; contacting a tumor cell with the cell death split-effector molecule; wherein (i) the split-effector polypeptide fragments are conjugated to probes specific for a particular target nucleic acid or target polypeptide that is associated with the cancer disorder; and wherein (ii) the split-effector polypeptide fragments combine to form an active effector molecule in the presence of a particular target nucleic acid or target polypeptide that is associated with the disease or disorder. In some embodiments, the use of the survival split-biomolecular conjugate as disclosed herein is useful for treatment of disease or disorder is associated with cell loss as part of the pathology, or a disease or disorder is associated with the expression of a pathological polypeptide, such as a mutated and/or incorrectly folded polypeptide or due to the expression of a pathological nucleic acid.

In some embodiments, a survival split-biomolecular conjugate as disclosed herein can is useful in the treatment of diseases such as neurological disease; kidney disease; cardiovascular disease; hepatic diseases; inflammatory diseases; cystic fibrosis; neurodegenerative diseases; inflammatory diseases; immune disorders, and neurological diseases such as Alzheimer's Disease, Huntington's disease; Parkinson's disease; amyotrophic lateral sclerosis (ALS), spinal cord injury.

Another aspect of the present invention, the split biomolecular conjugate is a proxy split-biomolecular conjugate comprising; a split-effector molecule, wherein the split-effector polypeptide fragments comprise at least two polypeptide fragments which are each conjugated to two or more probes, wherein the split-effector polypeptide fragments combine to form an active effector molecule in the presence of a particular target nucleic acid or target polypeptide that is capable of replacing a dysfunctional or lost polypeptide in the cell.

In some embodiments, a proxy split-biomolecular conjugate as disclosed herein is useful in methods for the treatment of a disease or disorder, comprising; contacting a tumor cell with the cell death split-effector molecule; wherein (i) the split-effector polypeptide fragments are conjugated to probes specific for a particular target nucleic acid or target polypeptide that is associated with the cancer disorder; and wherein (ii) the split-effector polypeptide fragments combine to form an active effector molecule in the presence of a particular target nucleic acid or target polypeptide that is associated with the disease or disorder, such as, for example a disease or disorder is associated with a loss or decreased expression or dysfunctional expression of a polypeptide within the cell.

In some embodiments, an effector polypeptide or a proxy split-biomolecular conjugate comprises the lost or dysfunctional polypeptide, or fragment thereof, associated with a disease, such as, for example a neurological disease; kidney disease; cardiovascular disease; hepatic diseases; inflammatory diseases; cystic fibrosis; neurodegenerative diseases; inflammatory diseases; immune disorders. Examples of such diseases include, for example, but are not limited to, muscular dystrophy, cystic fibrosis and the like.

Another aspect of the present invention relates to a pharmacological composition comprising at least one of the split biomolecular conjugates as disclosed herein, wherein the pharmaceutical composition comprises polymeric nanoparticles preloaded with at least one biomolecular conjugates.

### BRIEF DESCRIPTION OF FIGURES

FIG. 1 shows a schematic of targeted killing of pathological cells with a split toxin, and the nucleic acid based reassembly of a toxin for targeted killing of ALL cells. Two protein-oligonucleotide constructs with toxin halves linked to the RNA-recognition arms, which bind pathogenic RNA, are delivered into cells from the bloodstream by non-immunogenic drug-loaded vehicles (polymeric nanoparticles, cationic liposomes, etc.). Pathogenic RNA (endogenous oncogenic RNA or RNA of viral origin) present only in the diseased cells; it is thus a marker of these abnormal cells and serves as a scaffold for selective suicidal reassembly of a split toxin. To avoid possible degradation by intracellular nucleo-lytic enzymes, biostable oligonucleotide analogs can be used.

FIG. 2 shows a ribbon presentation of ricin A structure. Two major subdomains in the ricin A toxin (RTA) can clearly be recognized here: domain I formed by several β-sheets (arrows) and α-helical domain II.

FIG. 3 shows the structure of a TEL-AML1 gene fusion. (a) Schematic diagram of the exon/intron structure of the TEL and AML1 genes involved in (12;21)(p13;q22). The centromere (cen) and telomere (tel) orientation, exon numbering, and relevant breakpoint regions are indicated. (b) Schematic diagram of the TEL-AML1 fusion transcripts. The numbers under the fusion gene transcripts refer to the first (59) nucleotide of the involved exon, except when the last (39) nucleotide of the upstream gene is indicated. Most t(12;21)-positive patients have the larger transcript because of a breakpoint in AML1 intron 1, but alternative splicing can cause skipping of AML1 exon 2, leading to two PCR products products in some patients. In a minority of patients the AML1 breakpoint is located in intron 2, resulting in a shorter transcript without AML1 exon 2. The arrows indicate the relative position of the five primers; the numbers refer to the 59 nucleotide position of each primer (Van Dongen et al., 1999).

FIG. 4 shows the secondary structure predictions for the A chain of ricin (Predict Protein, H-helix, E-fl-sheet, L-Ioop) and suggested sites for protein splitting (arrows).

FIG. 5 shows as schematic of ricin fragments cloning and protein coupling with target-specific oligonucleotides (modified from burbulis et al, 2005). Panel 5A shows scheme of ricin A fragments cloned as fusions with interins, panel 5B shows the chemical structure of the modified oligonucleotide with pseudo-cystein at the 5′ end. Panel 5C shows a chemistry of coupling of the ricin fragment and modified oligonucleotide. Asterik denote a functional Cystein at the C-terminus of the ricin fragment, CBD-chitin binding domain.

FIG. 6 shows a structure of the breakpoint sequence for ALL.

FIG. 7 shows a schematic of the amino acid sequence of the full-length RTA (268 amino acids) and sites of initial split points. Panel 7A shows three split points (shown as ↓) of the RNA tested. The following considerations were taken into account to determine RTA split-points: (i) split point should separate the activity-important amino acids between the two protein halves; (ii) split point should be located within the unstructered region (to introduce a minimal structural disturbance to the split protein halves); (iii) split protein halves correspond to compact folding unit within the full-size RTA. Letters in BOLD ITALIC: active site (Y81, V82, G122, Y124, E178, R181,E209, N210 W212); BOLD UNDERLINED letters: amino acids crucial for RTA activity (R49, N79, N123, R214, R259); BOLD letters: amino acids with key structural role (D76, R135). Panel 7B shows shows the ribbon representation of the RTA structure. Two major subdomains of ricin A toxin (RTA) are shown: domain I formed by several β-sheets (arrows) (panel 7B) and α-helical domain II (panel 7C).

FIG. 8 shows complementing fragments of RTA gene obtained by PCR and purified by PCR-clean procedure to make the N-terminal fusions of split-RTA proteins to intein1 (subscript n is for N-terminal fusion). Estimated by gel electrophoresis sizes of these fragments correspond well to the expected ones: N1n 189 bp, N2n 396 bp, N3n 510 bp, C1n 681 bp, C2n 474 bp, C3n 360 bp (including primers carrying the restriction sites for subsequent cloning, stop codons, codons for terminal cystein, when necessary). After cloning in E. coli XL10 cells (cloning host), the identity of all PCR-amplified DNA fragments was additionally confirmed and verified by sequencing.

FIG. 9 shows SDS/PAGE protein expression patterns in the crude-cell preparations of E. coli BL21/DE3 expression host transformed with the mid-split RTA genes fused to intein1. Cells were induced (+) or not induced (−) with 1 mM IPTG at different temperatures for 2 hr (37° C.), 3 hr (30° C.) and 14 hr (25° C.). At all temperatures, induced cells exhibit considerable overexpression of N2n-RTA and C2n-RTA split RTA proteins. During subsequent experiments, the inventors discovered that induction at 30° C. is most appropriate for further optimization of protein expression.

FIG. 10 shows assessment of overexpressed split-RTA fusion proteins in soluble and insoluble fractions. IPTG induction was performed for 3 hr at 30° C. at low, 0.05 and 0.1 mM concentrations of inducer (N1n and C1n are only shown with 0.1 mM IPTG). In all panels, lanes 1 and 2 correspond to soluble and insoluble cellular protein fractions. Expected sizes of fusion proteins: N1n˜30 kDa; C1n˜50 kDa; N2n˜38 kDa; C2n˜40 kDa. The data demonstrates that the split RNA fragments frequently form inclusion bodies. The inventors have found that it is necessary to assess multiple different split sites in order to obtain two complementary halves of a split protein that do not form inclusion bodies. In particular, it may be necessary to test more than 4, or more than 5, or more than 6, or more than 7, or more than 8, or more than 9 or more than 10 different split points in a protein to find two complementary halves that are active when protein complemented together and inactive when they are alone.

FIG. 11 shows N1n-RTA obtained by refolding from inclusion bodies of N1n-RTA fusion with intein1 and subsequent self-splitting of the chitin column-bound intein. Single band of expected protein size ˜5.5 kDa is shown in the first lane, with total amount of N1n-RTA before concentrating approx ˜1 mg. N1c-RTA C-terminally fused to intein2 was also obtained (data not shown).

FIG. 12 shows Ricin splittable stem looped RNA oligonucleotide for testing functional reassembly of split RTA in vitro. An in vitro test system developed to analyze the activity of split/reassembled RTA proteins. Panel 12B shows RTA-splittable stem-loop RNA designed with dA in the loop region, 5′-r(GGAAUCCUGCUCAGUACG)-dA-r(GAGGAACCGCAGGUU) (SEQ ID NO:1), which accelerates the RTA action by depurination of this specific nucleobase with subsequent RNA cleavage by aniline treatment). Panel 12B shows the RNA is of a correct length for PAGE analysis and SYBR staining.

FIG. 13 shows a schematic of targeted killing of pathological cells with a split toxin, where each split-effector fragment protein and conjugated with a split-polypeptide probe that can associate with a nucleic acid probe, for example an aptamer. Two aptamers bind to the pathogenic RNA in the cells, allowing binding of the split-polypeptide probes which are conjugated to the split-effector fragments, allowing effector protein reassembly and formation of the active effector protein in the presence of pathogenic target nucleic acid such as pathogenic RNA. To avoid possible degradation by intracellular nucleo-lytic enzymes, biostable oligonucleotide analogs can be used.

FIG. 14 shows as schematic of for the split-effector fragment fused to a split-polypeptide probe. The split-effector fragments are N-terminal RTA fragments fused to CBD-intein or are C-terminal RTA fragments fused to CBD-intein.

### DETAILED DESCRIPTION

The inventors of the present invention have discovered a method for production and use of split-biomolecular conjugates for the targeted treatment of diseases, disorders and malignancies and screening. More specifically, the invention relates to methods to treat diseases, disorders and malignancies using a split-biomolecular conjugate comprising a split effector polypeptide, where each effector fragment is conjugated to a probe. Interaction of both probes with a target nucleic acid or target polypeptide, such as a pathogenic nucleic acid sequence or pathogenic protein, brings the effector fragments together to facilitate the reassembly, also referred to in the art as “protein complementation” of the effector molecule. Depending on the effector molecule, the protein complementation results in a cellular effect. The methods of this invention are based on therapeutic protein complementation methods. In one embodiment, one can label the target and monitor the need for and effectiveness of treatment, for example a treatment of a subject with a split-biomolecular conjugate as disclosed herein.

### DEFINITIONS

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention which can be had by reference to the specification as a whole. Accordingly, the terms defined immediately below are more fully defined by reference to the specification as a whole.

The term “split biomolecular conjugate” as used herein refers to a polypeptide comprising an effector molecule conjugated to a probe, where the effector molecule comprises fragments which are inactive as fragments, and are capable of reassembly or protein complementation to form a functional active effector molecule when attached probes interact with their specific target, typically a nucleic acid or polypeptide target.

The term “conjugate” as used herein refers to the attachment of two or more proteins joined together to form one entity. The proteins may attached together by linkers, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat sensitive linkers.

The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. As a non-limiting example, the nucleic acid sequence encoding one fragment of the split-biomolecular conjugate protein is derived from the nucleotide sequence of encoding one of the effector fragments fused in frame to an end, either the 5′ or the 3′ end, of a gene encoding a split polypeptide probe. In this manner, on expression of the gene, the effector fragment is functionally expressed and fused to the N-terminal or C-terminal end of the polypeptide probe. In certain embodiments, modification of the polypeptide probe is such that the functionality of the polypeptide probe remains substantially unaffected by fusion to the effector protein.

The term “linker” as used herein refers to any means to join two or more proteins by means other than the production of a fusion protein. A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling. It will be appreciated that modification which do not significantly decrease the function of the effector protein and/or the probe are preferred.

The term “effector molecule” as used herein refers to a polypeptide or a nucleic acid encoding a polypeptide, which when two fragments of an effector molecule come together, is functional, and has the capacity for the desired functional effect. As non-limiting examples, the effector molecules functional effect of the effector molecule can be to induce cell death, induce a cytotoxic effect, sensitize cells, degrade nucleic acids or polypeptides, promote cell survival, or to replace a lost and/or dysfunctional polypeptide etc.

The term “probe” used herein refers to a component conjugated to the fragment of the effector molecule of the split-biomolecular conjugate which recognizes and binds to target nucleic acid or target polypeptides and as a result of binding facilitates the protein complementation or reassembly of the conjugated split-effector molecules. In some embodiments, the probes may comprise nucleic acids or polypeptides.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide. The term “probe” as used herein refers to an oligonucleotide, polynucleotide, nucleic acid, either RNA or DNA, or nucleic acid analogue or polypeptide or probe, whether occurring naturally as in a purified restriction enzyme digest or produced synthetically, which is capable of annealing with or specifically hybridizing to a nucleic acid with sequences or polypeptides complementary to the probe. A nucleic acid probe may be either single-stranded or double-stranded, with the exact length of the probe depending upon many factors, including temperature, source of probe and the method used. For example, for diagnostic applications, depending on the complexity of the target sequence, the oligonucleotide probe typically contains 15-25 or more nucleotides, although it may contain fewer nucleotides. The probes herein are selected to be substantially complementary to target nucleic acid sequences or target polypeptides. This means that the probes must be sufficiently complementary so as to be able to “specifically hybridize” or anneal with their respective targets. Therefore, the probe need not reflect the exact complementary sequence or conformation of the target nucleic acid or polypeptide. For example, in the case of nucleic acid probes, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” as used herein refers to the association between two single-stranded nucleic acid molecules of sufficiently complementary sequence to permit such hybridization under pre-determined conditions generally used in the art (sometimes termed “substantially complementary”). In some embodiments, the term refers to hybridization of an oligonucleotide with a substantially complementary sequence contained within a single-stranded DNA or RNA probe of the invention, to the substantial exclusion of hybridization of the oligonucleotide with single-stranded nucleic acids of non-complementary sequence.

The term “nucleic acid” used herein refers to deoxyribonucleotides or ribonucleotides and polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term nucleic acid is used interchangeably with gene, cDNA, and mRNA encoded by a gene.

As used herein, the terms “oligonucleotide” and “primer” have the conventional meaning associated with it in standard nucleic acid procedures, i.e., an oligonucleotide that can hybridize to a polynucleotide template and act as a point of initiation for the synthesis of a primer extension product that is complementary to the template strand. Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

The term “conjugate” or “conjugated” as used herein refers to the joining of two or more entities. The joining can be fusion of the entities, for example fusion of polypeptides. Conjugation can also be performed by other means known in the art, for example but not limited to covalent, ionic, or hydrophobic interaction whereby the moieties of a molecule are held together and preserved in proximity.

The term “moieties” or “motif” is used interchangeably herein, refers to a molecule; nucleic acid or protein or polypeptide or otherwise, capable of performing a particular function. The terms “nucleic acid binding moieties” or “nucleic acid binding motif” refers to a molecule capable of binding to the nucleic acid in specific manner.

The term “pathogenic nucleic acid” or “pathogenic DNA” is used interchangeably herein, refers to the nucleic acid sequence that contributes, wholly or in part, to the symptoms, for example the structural and functional changes in cell, tissues and organs, which contribute to the disease disorder or malignancy.

The term “mutation” or “polymorphism” as used herein refers to a change in the nucleic acid sequence of nucleic acid, which can or can not affect the expression of the nucleic acid sequence. The term polymorphism is intended to include all polymorphisms, including deletions, substitutions, insertions, single nucleic acid polymorphisms (SNPs) etc. For example, mutations and polymorphisms may contribute to the disease disorder or malignancy, or alternatively may contribute to the responsiveness of a subject or cell to a therapy with particular pharmaceutical agents (this is often termed “pharmacogenomics”). Similarly, mutations and/or polymorphisms can identify subjects or cells which may not function correctly due to expression of a dysfunctional protein which is toxic to the cell, thus identifies subject and cells that have increased likelihood of developing a disease or disorder, or the cells or subject being responsive or not responsive to a treatment. In some embodiments, pharmacogenomics can also be used in pharmaceutical research to assist the drug development and selection process. (Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 3).

The term “regulatory sequences” and “regulatory elements” are used interchangeably herein, and refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence can comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, an optional operate sequence to control transcription, a sequence encoding suitable mRNA ribosomal binding sites, and sequences to control the termination of transcription and/or translation. In some embodiments, regulatory sequences can be selected for an assay to control the expression of split-biomolecular conjugate in a cell-type in which expression is intended. Regulatory sequences can be a single regulatory sequence or multiple regulatory sequences, or modified regulatory sequences or fragments thereof. In some embodiments, modified regulatory sequences are useful which are regulatory sequences where the nucleic acid sequence has been changed or modified by some means, for example, but not limited to, mutation, methylation etc.

As used herein, a “promoter,” “promoter region” or “promoter element” are used interchangeably herein, refers to a segment of a nucleic acid sequence, typically but not limited to DNA or RNA or analogues thereof, which controls the transcription of the nucleic acid sequence to which it is operatively linked. A promoter region can include specific sequences that are sufficient for RNA polymerase recognition, binding and transcription initiation, and this portion of the promoter region is referred to as the promoter. In addition, a promoter region can include sequences which modulate this recognition, binding and transcription initiation activity of RNA polymerase. In some embodiments, these sequences may be cis-acting or may be responsive to trans-acting factors. In some embodiments, promoters useful in the methods as disclosed herein and depending upon the nature of the regulation can be constitutive or regulated- or inducible-promoters.

The term “operatively linked” or “operatively associated” are used interchangeably herein refers to the functional relationship of the nucleic acid sequences with regulatory sequences of nucleotides, such as promoters, enhancers, transcriptional and translational stop sites, and other signal sequences. For example, a nucleic acid sequence, typically DNA, operatively linked to a regulatory sequence or promoter region, refers to the physical and functional relationship between the DNA and the regulatory sequence or promoter, such that the transcription of the linked DNA is initiated from the regulatory sequence or promoter, by a RNA polymerase that specifically recognizes, binds and transcribes the regulatory sequence. In some embodiments, order to optimize expression and/or in vitro transcription, it may be necessary to modify the regulatory sequence for the expression of the nucleic acid or DNA for expression of the cell type for which it is expressed. The desirability of, or need of, such modification may be empirically determined.

The term ‘nucleic acid binding motif’ as used herein refers to a region of a probe, nucleic acid or polypeptide capable of selectively binding to a nucleic acid sequence.

The terms “polypeptide,” “peptide” and “protein” are used interchangeably herein to refer to a polymer of amino acid residues. The terms apply to amino acid polymers in which one or more amino acid residue is an artificial chemical analog of a corresponding naturally occurring amino acid, as well as to naturally occurring amino acid polymers.

The term “pathogenic polypeptide” or “pathogenic peptide” or “pathogenic protein” are used interchangeably herein, refers to the polypeptide that contributes, wholly or in part, to a symptom of the disease disorder or malignancy.

The term “contributes substantially” in the context of “disease, disorder or malignancy” used herein is meant to refer to a pathological nucleic acids and/or pathological polypeptides which contribute, alone or with other nucleic acids and/or other polypeptides to the disease, disorder or malignancy.

The term ‘disorder’ or ‘disease’ used interchangeably herein, refers to any alteration in the state of the body or one of its organs, interrupting or disturbing the performance of and organ function (i.e. causes organ dysfunction) and/or causing a symptom such as discomfort, dysfunction, distress, or even death to a subject afflicted with the disease. In some embodiments, symptoms such as discomfort, dysfunction, distress, or even death can occur to subjects in contact with the subject with the disease. A disease or disorder can also relate to Distemper, ailing, ailment, malady, disorder, sickness, illness, complaint, indisposition, affection.

The term ‘malignancy’ and ‘cancer’ are used interchangeably herein, refers to diseases that are characterized by uncontrolled, abnormal growth of cells. Cancer cells can spread locally or through the bloodstream and lymphatic system to other parts of the body. Cancer diseases within the scope of the definition comprise benign neoplasms, dysplasias, hyperplasias as well as neoplasms showing metastatic growth or any other transformations like e.g. leukoplakias which often precede a breakout of cancer.

The term ‘toxin’ as referred to herein is intended to encompass any entity, typically a polypeptide which is capable of being cytotoxic, that is being toxic to a cell. The term “cytotoxin” as used herein refers to a toxic entity that is specifically toxic to a cell that is targeted to.

The term “immunotoxin” as used herein refers to a polypeptide comprising a toxic entity that is conjugated to a targeting entity to target specific cells. In some embodiments, a targeting entity is the probe component of the split-biomolecular conjugate.

The term “nuclease” or “endonuclease” or “exonuclease” are used interchangeably herein, refers to molecules capable of degrading nucleic acid sequences into small nucleic acid sequences or single nucleotides.

The term “oncogene” as used herein refers to a nucleic acid sequence encoding, or polypeptide, of a mutated and/or overexpressed version of a normal gene that in a dominant fashion can release the cell from normal restraints on growth. Oncogenes can alone or in concert with other changes or genes, contribute to a cells tumorigenicity. Examples of oncogenes include; gp40 (v-fms); p21 (ras); p55 (v-myc); p65 (gagjun); pp60 (v-src); v-abl; v-erb; v-erba; v-fos etc. A “proto-oncogene” or “pro-oncogene” refers to the normal expression of a nucleic acid expressing the normal, cellular equivalent of an oncogene, typically these genes are usually a gene involved in the signaling or regulation of cell growth.

The term “sensitize” or “sensitizes” are used interchangeably herein, refers to making the cell sensitive, or susceptible to other secondary agents, for example other pro-drugs or other environmental effects such as radiation etc.

The term “cell” as used herein refers to any cell, prokaryotic or eukaryotic, including plant, yeast, worm, insect and mammalian. Mammalian cells include, without limitation; primate cells, human cells and a cell from any animal of interest, including without limitation; mouse, hamster, rabbit, dog, cat, domestic animals, such as equine, bovine, murine, ovine, canine, feline and transgenic animals etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, neural, stromal, muscle, cardiovascular, hepatic, pulmonary, gastrointestinal stem cells, etc. Yeast cells may also be used as cells in this invention. Cells also refer not to a particular subject cell but to the progeny or potential progeny of such a cell because of certain modifications or environmental influences, for example differentiation, such that the progeny mat not, in fact be identical to the parent cell, but are still included in the scope of the invention.

The cells used in the invention can also be cultured cells, e.g. in vitro or ex vivo. For example, cells cultured in vitro in a culture medium, Alternatively, for ex vivo cultured cells, cells can be obtained from a subject, for example a healthy subject and/or a subject affected with a disease. Cells can be obtained, as a non-limiting example, by biopsy or other surgical means know to those skilled in the art. Cells used in the invention can present in a subject, e.g. in vivo. For the invention on use on in vivo cells, the cell can be is found in a subject and display characteristics of the disease, disorder or malignancy pathology.

As used herein, the term “subject” is intended to include human and non-human animals. The term “non-human animals” includes all vertebrates, e.g. mammals, non-mammals, such as non-human primates, sheep, dog, cow, chickens, amphibians, reptiles, rodents etc. In certain embodiments, the subject is mammal, e.g., a primate, e.g., a human.

As used herein, the term “pathogen” refers to an organism or molecule that causes a disease or disorder in a subject, for example, pathogens include but are not limited to viruses, fungi, bacteria, parasites and other infectious organisms or molecules therefrom. In some embodiments, viruses can be selected from a group of viruses comprising of Herpes simplex virus type-1, Herpes simplex virus type-2, Cytomegalovirus, Epstein-Barr virus, Varicella-zoster virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B. Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Simian hnmunodeficiency cirus, Human T-cell Leukemia virus type-1, Hantavirus, Rubella virus, Simian Enmunodeficiency virus, Human Immunodeficiency virus type-1, and Human Immunodeficiency virus type-2.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. In some embodiments, a mammal is a human.

The term “genetic predisposition” as used herein refers to the genetic makeup of a subject or cell, that makes predetermines the subject or cells likelihood of being susceptible to a particular disease, disorder or malignancy, or likelihood of responding to a treatment for a disease disorder or malignancy.

The term “cell death pathway” as used herein refers to pathway of the cell suicide pathway or programmed cell death program (PCD), also known as apoptosis, which is well known by persons skilled in the art. ‘Anti-apoptosis’ and ‘pro-apoptosis’ as used herein refer to molecules or entities which prevent or induce the cell death pathway respectively.

The term “humanized” used herein refers to a nucleic acid sequence or polypeptide which has been modified, either genetically or post-transcriptionally to form of nucleic acid or polypeptide that has been optimized for expression and function in mammalian cells.

The term “amplification” primers as used herein refer to oligonucleotides comprising either natural or analog nucleotides that can serve as the basis for the amplification of a select nucleic acid sequence. They include, e.g., polymerase chain reaction primers and ligase chain reaction oligonucleotides.

The term “recombinant” when used in reference to, for example, a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (non-recombinant) form of the cell or express native genes that are otherwise abnormally expressed, under expressed or not expressed at all.

The term “contacting” as used herein refers to the placement in direct physical association. With regards to this invention, the term refers to antibody-antigen binding.

The term “cytotoxicity”, as used herein, typically refers to directed inhibition of normal cellular function of a selected or targeted cell, for example in some instances cytotoxicity refers to the inhibition of protein synthesis. Such inhibition of protein synthesis can be assayed in human tumor cells, e.g., HS578T (ATCC No. HTB 126) using the protocol described in Rybak, et al., JNC_{1-88:747}-753 (1996). A “cytotoxic reagent” as used herein can have a relative 50% inhibitory concentration (IC₅₀) at least 50% that of an equimolar amount of the polypeptide. In some instances, the relative IC₅₀ will be at least 60% or 70% that of the polypeptide, or at least 100%.

In some embodiments, in order for a particular cell to express the proteins encoded by nucleic acid sequences, the nucleic acid can be introduced into the cell using any method commonly known by persons of ordinary skill in the art. Methods to introduce DNA into cells include, but are not limited to transformation by an appropriate vector. The term “transformation” as used herein refers to the introduction of heterologous polynucleotide or nucleic acid sequence or fragment thereof into a host cell, using any known method in the art, for example, but not limited to direct uptake, transfection or transduction. In some embodiments, for the production of the split-biomolecular conjugate as disclosed herein, a cell can be transformed with at least one nucleic acid construct, wherein one construct comprises the sequence for at least one fragment of the split-biomolecular conjugate as disclosed herein, where the cell then expresses the nucleic acid encoding the split-biomolecular conjugate to produce the split-biomolecular conjugate protein. The construct can be introduced into the cell by multiple means known to persons skilled in the art, including vectors, viral vectors, and non-viral means, such as, but not limited to non-viral means such as fusion, electroporation, biolistics, transfection, lipofection, protoplast fusion, calcium phosphate transfection, microinjection, pressure-forced entry, naked DNA etc., or any other means known any persons of ordinary skilled in the art.

The term “vectors” and “plasmid” are used interchangeably herein, refers to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in the methods as disclosed herein include recombinant DNA techniques using “plasmids” which refer to circular double stranded DNA loops which, in their vector form are not bound to the chromosome. Other expression vectors can be used in different embodiments for example, but are not limited to, plasmids, episomes, bacteriophages or viral vectors, and such vectors may integrate into the host's genome or replicate autonomously in the particular cell. Other forms of expression vectors known by those skilled in the art which serve the equivalent functions can also be used. Expression vectors comprise expression vectors for stable or transient expression encoding the DNA.

### Effector Molecules

### Cytotoxic Effector Molecules

In some embodiments, an effector molecule may be a cytotoxin, for example a bacterial toxin or bacterial cytotoxin. Bacterial toxins or cytotoxins are well known to persons skilled in the art for example but not limited to anthrax toxin; diphtheria toxin (DT); ricin A toxin (RTA); pseudomonal endotoxin (PE); streptolysin O; saporin; gelanin or naturally occurring variants, or genetically engineered variants or fragments thereof. Bacterial toxins are typically not glycoslyated, but glycosylated bacterial toxins are also encompassed for use in the invention. For example, DT has been genetically modified to improve their specificity and non-specific binding to normal cells, for example DT is mutated by converting leu 390 and Ser525 each to phenylalanine, resulting in CRM107 (Greenfield et al; Sci, 1987; 238:536-539), or DT and PE, including PE40, have been truncated (Francisco et al; 1997; 272:24165-24169; Kondo et al, 1988; 263:9470; Williams et al, 1987; 1:493-498). For variations of mutations and modifications to modify the properties of bacterial toxins used, see the review; Immunotoxins for Targeted Cancer Therapy; Kreitman R. J, 2006; APPS Journal; 8(3);E532-E551, incorporated in its entirety herein for reference.

In another embodiment, effector molecule can be a plant toxin. Plant toxins are well known to persons skilled in the art and can be a plant halotoxin or class II ribosome inactivating protein, or a hemitoxin or class I ribosome inactivating protein. A plant halotoxin can be for example, but not limited to saporin (SAP); pokeweed antiviral protein (PAP); bryodin 1; bouganin and gelonin or naturally occurring variants, or genetically engineered variants or fragments thereof. A plant hemitoxin can be, for example ricin A chain (RTA); ricin B (RTB); abrin; mistletoe, lectin and modeccin or naturally occurring variants, or genetically engineered variants or fragments thereof. Plant toxins are typically glycoslyated, but non-glycosylated plant toxins are also encompassed for use in the invention.

In alternative embodiments, the plant toxins can function as nucleases, for example, but not limited to sarcin; restrictocin.

In alternative embodiments of the invention, the effector molecule can comprise the polypeptide or fragment of a cytotoxic molecule or protein. One example of a cytotoxic molecule is cytokine. Non-limiting examples of cytokines that have been used as toxins for cancer include IL-1; IL-2 (CD25); IL-3; IL-4; IL-13; interferon-alpha; tumor necrosis factor-alpha (TNFα); IL-6; granulocyte-macrophage colony stimulating factor (GM-CSF); G-CSF. The cytokines can be or natural occurring variants of cytokines or alternatively been genetically engineered variants thereof, or cytokines comprising a heterologous sequence of recombinant cytokines.

In an alternative embodiment, the effector molecule can comprise humanized immunotoxins, that comprise a human or mammalian toxin, for example but not limited to RNase, protamine/DNA, and Bax. For review of examples of humanized toxins, see review by Frankel, A., Clinical Cancer Res, 2004; 10:13-15, which is incorporated herein in its entirety by reference.

### Nucleases Effector Molecules

In another embodiment, an effector molecule can have nuclease or endonucleolytic activity. In some embodiment the nuclease is a DNA nuclease, DNA endonuclease, or DNA exonuclease. The nuclease can be a natural variant, homologue or a genetically modified variant thereof. Examples of known DNA endonucleases are well known to persons skilled in the art, and have been used for conjugates for immunotoxins (see WO0174905, which is incorporated herein in its entirety by reference, and include examples, such as bovine DNaseI (see Worrall and Conolly, 1990; J. Biol. Chem. 265; 21889-21895); pancreatic DNaseI (see Shak et al, 1990; Proc. Natl. Acad. Sci. USA., 87; 9188-9192 and Hubbard et al, 1992; New Eng. J. Med., 326:812-815). In some embodiments, the DNase nuclease is a mammaliand deoxyribonuclease I, and in others it is a human deoxyribonuclease I.

In an alternative embodiment of the invention, the nuclease is a RNA nuclease, RNA endonuclease or RNA exonuclease. RNA nucleases are well known to persons skilled in the art, any of which are encompassed for use in this invention. Non-limiting examples of RNA nucleases include RNA endonuclease I; RNA endonuclease II; RNA endonuclease III. In some instances of the invention, the RNase can be a ribonuclease A (RNase A), one such example is referred to the trade name of Onconase®, (available from Alfacell Corporation, Bloomingfield, N.J.) derived from Rana pipens oocytes that was originally designated P-30 and first described in Darzynkiewicz et al, Cell Tissue Kinet, 21; 169 (1998). One of skill will appreciate any RNase or RNase A molecule can be modified using numerous methods known to those skilled in the art, and use of such modified or recombinant RNase and/or RNase A molecules, or naturally occurring variants thereof, as effector proteins are encompassed for use in the methods as disclosed herein. In some embodiments, RNase A can be used as an effector molecule which has been disclosed in the use as an immunotoxin in European Patent Application EP975671; US Patent Application U.S. Pat. No. 6,869,604, which are incorporated herein by reference, which use ribonuclease derived from Rana pipiens. In other embodiments, ribonucleases derived from Rana catesbeiana oocytes can be used as effector molecules. Although the amino acid sequence of Rana catesbeiana oocyte RNAse (RaCOR1) has been known since 1989 (Nitta, R., et al., J. Biochem. 106:729 (1989); Okabe, Y., et al., J. Biochem. 109:786 (1991); Liao, Y, Nuc., Acids Res. 20:1371 (1992); Nitta, K., et al., Glycobiology 3:37 (1993); Liao, Y. & Wang, J., Eur. J. Biochem. 222:215 (1994); Wang, J., et al., Cell Tissue Res. 280:259 (1995); Liao, Y., et al., Protein Expr. Purif. 7:194 (1996); and Inokuchi, N., et al., Biol. Pharm. Bull. 20:471 (1997)), genomic DNA or mRNA which encodes oocyte RNAses and genetically modified variants thereof are also encompassed.

In another related embodiment, RNases useful in the methods as disclosed herein can be of the superfamily of human pancreatic RNases, for example human angiogenin or a fragment thereof, or a recombinant or genetically engineered variant thereof having ribonuclease activity (Kurachi et al, 1985; Biochemistry, 24; 5494-5499. Angiogenin is also a potent inhibitor of protein synthesis in cell-free extracts and upon injection into Xenopus oocytes. Extracellular angiogenin is not cytotoxic towards a wide variety of culture cells and is normally present in human plasma, therefore its reconstitution within a cell is an ideal candidate as an effector molecule in the split-biomolecular conjugate. Further, human angliogenin has been used in immunotherapy by conjugating to IL2, see European Patent Application EP1217070, incorporated herein in its entirety for reference, and has also been shown to be expressed as two portions of two human proteins or fragments thereof (see European Patent Application EP1217070).

In another embodiment of the invention, an RNase can be Dicer. Dicer or Dcr-1 homolog (Drosophila) is a RNase III nuclease that cleaves double-stranded RNA (dsRNA) and pre-microRNA (miRNA) into short double-stranded RNA fragments of about 20-25 nucleotides long, usually with a two-base overhang on the 3′ ends (often called small interfering RNA (siRNA)). Because dicer contains two RNase domains and one PAZ domain; an effector molecule could comprise each domain of Dicer. Dicer catalyzes the first step in the RNA interference pathway and initiates formation of the RNA-induced silencing complex (RISC), whose catalytic component argonaute is an endonuclease capable of degrading messenger RNA (mRNA) whose sequence is complementary to that of the siRNA guide strand.

In other embodiments, the nuclease is a restriction endonuclease, for example microbial type II restriction endonucleases. Exemplary but non-limiting examples of type II restriction endonucleases include; BamHI; Hind III; MspI; Sau3AI; Hinfl; NotI; and EcoRI.

### Other Effector Molecules

A further embodiment, and effector molecule can be a proteolytic enzyme, or protease molecule (also known as proteinases, peptidases, or proteolytic enzymes) which break peptide bonds between amino acids of proteins by a process called proteolytic cleavage and is a common mechanism of activation or inactivation of enzymes. Some proteases use a molecule of water for proteolytic cleavage and are also classified as hydrolases. Proteases useful in the methods as disclose herein are well known by persons skilled in the art, and include for example, but are not limited to, serine proteases; threonine proteases; cysteine proteases; aspartic acid proteases (e.g., plasmepsin); metalloproteases; glutamic acid proteases; endopeptidases (proteinases) and exopeptidases. Common proteases are, for example; caspase enzymes; calpain enzymes; cathepsin enzymes; endoprotease enzymes; granzymes; matrix metalloproteases; pepsins; pronases; proteases; proteinases; rennin; trypsin, and their use, or naturally occurring homologues or genetically engineered variants thereof are encompassed for use in this invention.

In another embodiment, an effector molecule can be any molecule capable of inducing a cell death pathway in a cell. Examples of such effector molecules include, but are not limited to, pro-apoptotic molecule which are well known in the art, for example but not limited to Hsp90; TNFα; DIABLO; BAX; BID; BID; BIM; inhibitors of Bcl-2; Bad; poly ADP ribose polymerase-1 (PARP-1); Second Mitochondria-derived Activator of Caspases (SMAC); apoptosis inducing factor (AIF); Fas (also known as Apo-1 or CD95); Fas ligand (FasL) are encompassed for use as effector molecules by the methods as disclosed herein, as well as natural variants or recombinant or genetically modified variants of such pro-apoptotic molecules.

In alternative embodiments, an effector molecule useful in the methods as disclose herein is capable of inhibiting a cell death pathway or inducing a cell survival pathway in the cell. Examples of such molecules include, but are not limited to numerous anti-apoptotic molecules which are well known by person of ordinary skill in the art, for example but not limited to; Bcl-2; Bcl-XL; Hsp27; inhibitors of apoptosis (IAP) proteins.

In another embodiment of the invention, an effector molecule can be a molecule or polypeptide that sensitizes the cell to one or more secondary agents. For example, an effector molecule can be a tyrosine kinase, for example β glucuronidase activity. β-Glucuronidase activates the low-toxic prodrugs such as 9-aminocamptothecin and p-hydroxy aniline mustard, or analogue such as a N-[4-doxorubicin-N-carbonyl(oxymethyl)phenyl]O-β-glucuronyl carbamate (DOX-GA3) have been developed to improve the antitumor effects of doxorubicin (DOX). The prodrug DOX-GA3 was initially designed to be activated into an active molecule or drug by human β-glucuronidase (GUS) to result in a highly cytotoxic effect specifically in the tumor site. The potency of such prodrugs can also be greatly enhanced with the incorporation of an appropriate radionuclide in a combined chemo- and radio-therapy of anti-cancer (CCRTC) strategy. In some embodiments, the prodrug can also be utilized to modify liposomes for efficient delivery of anti-cancer drugs (Chen et al, current medicinal chemistry; 2003 3; 139-150; Chen et al, cancer Gene Ther, 2006).

In another embodiment, an effector molecule that sensitizes the cell to another agent is, for example, hypoxanthine-guanine phosphoribosyltransferase (HGPRT), from the parasite Trypanosoma brucei (Tb), which can convert allopurinol, a purine analogue, to corresponding nucleotides with greater efficiency than its human homologue, therefore is capable of activating the prodrug allopurinol to a cytotoxic metabolite (Trudeau et al, 2001; Human Gene Ther; 12:1673-1680). In another embodiment, the effector molecule can be the bacterial nitrobenzene nitroreductase (NbzA) from Pseudomonas pseudoalcaligenes JS45, which activates the dinitrobenzamide cancer prodrug CB 1954 and the proantibiotic nitrofurazone (Berne et al, 2006; Biomacromolecules, 7; 2631-6).

In another embodiment, an effector molecule is β-lactamase, which produces active agents or drugs from the pro-drug desacetylvinblastine-3-carboxylic acid hydrazide (DAVLBHYD) or other analogues. In such and embodiment, the Enterobacter cloacae beta-lactamase (bL) as an effector protein can activate the anticancer prodrugs 7-(4-carboxybutanamido)cephalosporin mustard (CCM), a cephalosporin prodrug of phenylenediamine mustard (PDM) (Svensson et al, 1999; J Med. Chem., 41:1507-12). Other prodrug/enzyme combinations known in the art can be used as the effector molecule and are encompassed for use in the methods as disclosed herein, including enzymes that produce toxic radicals on photodynamic therapy (see wardman et al, 2001; scientific yearbook, 2001-2002), for example peroxidase genes can be used as effector molecules.

In a related embodiment, an effector molecule can be a molecule that catalyzes an antiviral drug, for example, but not limited to Oseltamivir which is commonly used as an anti-viral drug can act as a secondary agent for carboxylesterase HCE1 as an effector molecule (Shi et al, 2006; J Pharmacol Exp Ther.)

In some embodiments of the invention, an effector molecule can initiate addition or modification of a target nucleic acid or target polypeptide molecule. As a non-limiting example, where the target is a target polypeptide, a useful effector molecule can be ubiquitin, which adds, by covalent attachment, one or more ubiquitin monomers and tag the target polypeptide to be degraded via the proteasome. An example of another embodiment where the target is a polypeptide, the effector molecule could a Small Ubiquitin-related Modifier (SUMO) which tags the target polypeptide for numerous effects, including increased polypeptide stability, cellular localization etc. Other post-transcriptional events are known to persons skilled in the art, and include for instance; ISGylation; acetylation, alkylation, methylation biotinylation, glutamylation, glycylation; glycosylation, isoprenylation, lipoylation, phosphopantetheinylation, citrullination; deamidation, phosphorylation, etc., and the molecules that mediate or affect these events can be used as effector molecules in the methods as disclosed herein.

In another embodiment, where the target is a target nucleic acid, an effector molecule useful in the methods as disclosed herein can modify the nucleic acid, for example chemical modification, includes, for example methylation or structural modification, for example acetylation or addition of histones to silence the gene and/or to prevent the transcription of the target nucleic acid. In one embodiment, an effector molecule can be a DNA methyltransferase (DNA MTase), for example, DNMT1, DNMT2, DNMT3A, DNMT3B or de novo methyltransferases or fragments thereof which will methylate the target nucleic acid on protein complementation. In another embodiment, an effector molecule is a histone acetyltransferase enzymes (HATs), such as CREB-binding protein, or modified version or variant thereof.

### Probes

In some embodiments, a probe of the split-biomolecular conjugate can be any molecule that is capable of binding to a target nucleic acid. The region of the probe that binds to the target nucleic acid is referred to a nucleic acid binding motif. In some such embodiments, a probe useful in the methods as disclosed herein includes nucleic acids, nucleic acid analogues, and polypeptides. In one embodiment, a probe is an oligonucleotide. In some embodiments, a pair of probes of split-biomolecular conjugate can be the same kind of molecule, for example both probes can be oligonucleotides, or they can be different, for example one probe of the split-biomolecular polypeptide pair can be an oligonucleotide probe, and the other probe of the corresponding split-bimolecular polypeptide pair can be a polypeptide probe.

In some embodiments, the probe can be any molecule that can be coupled to another molecule, which is capable of binding to a target nucleic acid or target polypeptide in close proximity. In some embodiments, a probe can be a nucleic acid or nucleic acid analogue, such as an oligonucleotide. In another embodiment a probe can be a nucleic-acid binding polypeptide or proteins, which interacts with the target nucleic acid or target polypeptide with high affinity. Probes that are nucleic acid analogues include, for example but not limited to, peptide nucleic acids (PNAs), pseudocomplementary PNA (pcPNA), locked nucleic acids (LNA), morpholin DNAs, phosphorthioate DNAs, and 2′-O-methoxymethyl-RNAs.

In some embodiments, probes can bind to the same hybridization site on a single-stranded target, creating a triplex at the hybridization site comprising the target nucleic acid, two probes hybridizing the same site. Alternatively, probes can bind to closely adjacent hybridization sites on a single-stranded or double-stranded target nucleic acid, creating either a duplex or a triplex at each hybridization site, respectively.

In some embodiments, where probe is a nucleic acid, the length of the nucleic acid binding motif can be long enough to allow complementary binding to the nucleic acid target or polypeptide target, and allows one of the split-biomolecular conjugate fragments to interact with its corresponding split-biomolecular conjugate fragment(s) when both probe portions are bound to the same target nucleic acid or target polynucleic acid. For example, the nucleic acid binding moiety probe can be 5-30 bases long or in other embodiments, a nucleic acid probe can be 5-15 bases long. For example, 5, 6, 7, 8, 9, 10, 11, 12, 13, 14, 15, 16, 17, 18, 19, 20, 21, 22, 23, 24, 25, 26, 27, 28, 29, or 30 bases. In certain alternative embodiments, the probe can be than 30 bases long.

In some embodiments providing for formation of a triplex, a probe can be any nucleic acid which allows triplex formation. In some embodiments, triplex-forming oligonucleotides are GC-rich, for example a purine triplex, consisting of pyrimidine-purine-purine.

In some embodiments, a nucleic acid probe can be for example, but not limited to oligonucleotides; single stranded RNA molecules; and peptide nucleic acids (PNAs) including pseudocomplementary PNAs (pcPNA) etc. In some embodiments, a probe is an oligonucleotide. Methods for designing and synthesizing oligonucleotides are well known in the art. Oligonucleotides are sometimes referred to as oligonucleotide primers. Oligonucleotides useful in the methods as disclosed herein can be synthesized using established oligonucleotide synthesis methods, which are well known by persons of ordinary shill in the art. Such methods can range from standard enzymatic digestion followed by nucleotide fragment isolation (see for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference), to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method).

In some embodiments, oligonucleotides probes or nucleic acid probes useful in the methods as described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

In some embodiments, the probes can be are single stranded RNA molecules, and designed and synthesized using methods for single stranded RNA molecule production which are well known by persons of ordinary skill in the art.

In alternative embodiments, probes can be a nucleic acid binding moieties such as peptide nucleic acids (PNAs), including pseudocomplementary PNAs (pcPNA). Methods for designing and synthesizing PNAs and pcPNAs are well known by persons of ordinary skill in the art. Peptide nucleic acids (PNAs) are analogs of DNA in which the backbone is a pesudopeptide rather than a sugar. Thus, their behavior mimics that of DNA and binds complementary nucleic acid strands. In peptide nucleic acids, the deoxyribose phosphate backbone of oligonucleotides has been replaced with a backbone more akin to a peptide than a sugar phosphodiester. Each subunit has a naturally occurring or non naturally occurring base attached to this backbone, for example a backbone can be constructed of repeating units of N-(2-aminoethyl)glycine linked through amide bonds.

PNA binds to both DNA and RNA to form a PNA/DNA or PNA/RNA duplexes which bind with greater affinity and increased specificity than corresponding DNA/DNA or DNA/RNA duplexes. In addition, the polyamide backbone of PNA (having appropriate nucleobases or other side chain groups attached thereto) is not recognized by either nucleases or proteases, and thus PNAs are resistant to degradation by enzymes, unlike DNA and peptides. The binding of a PNA strand to a DNA or RNA strand can occur in either a parallel of anti-parallel orientation. PNAs bind to both single stranded DNA and double stranded DNA.

In some embodiments, pseudocomplementary PNAs (pcPNAs) can be used which are a variation of PNA molecules and includes, in addition to guanine and cytosine, pcPNA's carry 2,6-diaminopurine (D) and 2-thiouracil instead of adenine and thymine, respectively. pcPNAs exhibit a distinct binding mode, double-duplex invasion, which is based on the Watson-Crick recognition principle supplemented by the notion of pseudocomplentarity. pcPNAs recognize and bind with their natural A, T, (U), or G, C counterparts. pcPNAs can be made according to any method known in the art. For example, methods for the chemical assembly of PNAs are well known (See: U.S. Pat. No. 5,539,082, 5,527,675, 5,623,049, 5,714,331, 5,736,336, 5,773,571 or 5,786,571 which are incorporated herein by reference).

In some embodiments, the probe can be a polypeptide which are referred to as “polypeptide detector proteins” herein. In some embodiments, a polypeptide detector protein can be any polypeptide with a high affinity for the target nucleic acid or target polypeptide. In some embodiments, a target nucleic acid can be a double-stranded, triple-stranded, or single-stranded DNA or RNA. In some embodiments, a polypeptide probe is a peptide, less than 100 amino acids, or a full length protein, or a protein fragment. In some embodiments, a polypeptide's affinity for the target nucleic acid can in the low nanomolar to high picomolar range. Polypeptides useful in the methods as disclosed herein include polypeptides which contain zinc fingers, either natural or designed by rational or screening approaches. Examples of zinc fingers include Zif 2g8, Sp1, finger 5 of Gfi-1, finger 3 of YY1, finger 4 and 6 of CF2II, and finger 2 of TTK (PNAS (2000) 97: 1495-1500; J Biol Chem (20010 276 (21): 29466-78; Nucl Acids Res (2001) 29 (24):4920-9; Nucl Acid Res (2001) 29(11): 2427-36). Other polypeptides which are useful in the methods as disclosed herein include polypeptides, obtained by in vitro selection, that bind to specific nucleic acids sequences, for example, aptamers such as aptamers of platelet-derived growth factor (PDGF) (Nat Biotech (2002) 20:473-77) and thrombin (Nature(1992) 355: 564-6. Other polypeptides useful in the methods as disclosed herein include polypeptides which bind to DNA triplexes in vitro; for example, members of the heteronuclear ribonucleic particles (hnRNP) proteins such as hnRNP K, L, E1, A2/B1 and I (Nucl Acids Res (2001)₂₉(11): 2427-36).

In some embodiments, where a split-biomolecular conjugate fragment comprise polypeptides as the probes, the entire split-polypeptide fragment and probe can be encoded by a single nucleic acid construct comprising nucleic acid encoding polypeptide effector protein fragment, a linker sequence and the nucleic acid sequence encoding the nucleic acid binding moiety polypeptide or polypeptide detector protein. In some embodiments, a polypeptide detector protein in a cell or microinjected into a cell. In some embodiments, such constructs can also be used for in vitro detection of a nucleic acid of interest.

In some embodiments where the probe is a polypeptide detector protein, the polypeptide detector protein can be split into at least two fragments, wherein each fragment is conjugated to at two or more fragments of the effector protein, and wherein the binding of the detector polypeptide fragments to the target nucleic acid or target polypeptide reconstitutes the detector protein and the active effector protein. For example, a polypeptide probe could be a detector protein that contains multiple domains (for example zinc finger motifs) or a nucleic acid binding molecule which has been split into two separate components, such as eIF-4A (see Patent Application 60/730,746 which is incorporated herein its entirety for reference). In some embodiments, where the detector polypeptide probe is a multi-domain polypeptide detector protein, each domain can be conjugated to at two or more fragments of the effector protein, and where upon the binding of the domains of the detector protein to the target nucleic acid or target polypeptide results in reconstitution of the detector protein and the active effector protein.

### Target Nucleic Acid and Target Polypeptides

One aspect of the present invention is recognition of target nucleic acids or target polypeptides by the split-biomolecular conjugate as disclosed herein, which comprises an split-effector molecule where each fragment is conjugated to a probe. In one embodiment, the probe recognizes a target nucleic acid, and in another, the probe recognizes a target polypeptide.

In some embodiments, the target nucleic acid is DNA or RNA. In some embodiments, a target nucleic acid is a pathological nucleic acid (DNA or RNA) or pathology causing nucleic acid, for instance, the pathological nucleic acid is the nucleic acid that contributes substantially the disease, disorder or malignancy. This includes but is not limited to, for example, nucleic acid sequences encoding a mutation and/or polymorphism in a gene; regulatory sequence operatively linked to a gene or in the 5′ or 3′ untranslated regions (UTR) of a gene. In some embodiments, a pathological nucleic acid is a nucleic acid sequence that expresses a gene product that contributes in part, or wholly to a disease, disorder or malignancy, for example, genes that are expressed constitutively (i.e. permanently), such as active like the epidermal growth factor receptor (EGFR), platelet-derived growth factor receptor (PDGFR), and vascular endothelial growth factor receptor (VEGFR) and HER2/neu. The gene product may be a mutant or normal polypeptide.

In some embodiments, a target nucleic acid or target polypeptide is a gene or gene product that is expressed in a malignant cell. As an exemplary example, a target nucleic acid is the nucleic acid encoding polymorphic epithelial mucin (PEM), a compontent of the human milk fat globule that is expressed in cells in several body tissues and also in urine and is known to be expressed in epithelial cancer cells, notably ovarian, gastric, colorectal and pancreatic cancer cells. In such an embodiment, a target nucleic acid is a nucleic acid sequence encoding of the polymorphic epithelial mucin (PEM), and/or a target polypeptide is an antigen of PEM or cytotoxic portion of PEM. In some embodiments the probe targets PEM similar to being targeted by immunotoxins, as disclosed in WO0174905, which is incorporated herein by reference. In some embodiments, a target nucleic acid or target polypeptide can be an oncogene or an oncogenic molecule or oncogene or receptor kinase signaling molecule. In some embodiments, a target nucleic acid encodes an angiogenesis protein, for example but not limited to vascular endothelial growth factor (VEGF) or VEGF-1 or homologues thereof.

In some embodiments, a pathological nucleic acid is pathogenic DNA or RNA, for example but not limited to viral genomic sequences such as from hepatitis type A, hepatitis type B, hepatitis type C, influenzia, varicella, adenovirus, HSV-1, HSV-II, rinderpest rhinovirus, echovirus, retroviruses, rotavirus, respiratory syncytial virus, papilloma virus, papova virus, cytomegalovirus, echinovirus, abovirus, hantavirus, coxsackie virus, mumps virus, measles virus, rubella virus, polio virus, HIV-1, HIV-II, SARS, avian and/or bird flu viruses and other viruses or variants thereof.

In some embodiments, a pathological polypeptide is a target to the split-biomolecular conjugate as disclosed herein, where the polypeptide contributes to part, or wholly, a symptom of a disease, disorder or malignancy. In some embodiments, a pathological polypeptide is any protein that contributes to a symptom of a disease due to dysfunctional or abnormal expression. For example, but not limited to, a pathogenic polypeptide can be a protein that is mutated and/or unfolded and/or in the abnormal conformation, and/or in the incorrect subcellular localization and/or expressed in inappropriate cell and tissue types or inappropriate or lack of association with other proteins. As an illustrative example only, a pathogenic polypeptide can be a protein that contributes to a symptom of a disease such as a cancer, for example such pathogenic polypeptide can be an angiogenesis protein such as EGF and VEGF, or contribute to neurodegenerative diseases such as β-amyloid in Alzheimer's disease; mutant SOD1 in amyotrophic lateral sclerosis (ALS) etc.

In some embodiments, a pathological polypeptide can be a polypeptide expressed on the surface of a pathogen, for example polypeptides that form part of the coat protein or caspid of virus particles, as a non-limiting example, the gp40 expressed on HIV virus particle, or other surface markers expressed on cancer cells, viruses or infectious particles.

Uses of the Biomolecular Conjugate

In some embodiments, a biomolecular conjugate as disclosed herein can be used to trigger cell death. In such embodiments, the split-biomolecular conjugate is referred to as a “cell death split-biomolecular conjugate” and comprises a split-effector molecule which when complemented to be active is capable of activating cell death. For example, wherein the split-effector polypeptide fragments comprise at least two polypeptide fragments which are each conjugated to two or more probes, wherein the split-effector polypeptide fragments combine to form an active effector molecule in the presence of a particular target nucleic acid or target polypeptide that is capable of initiating a cell death pathway in the cell. In such an embodiment, the effector molecule can be a toxin, cytotoxic molecule, nuclease, proteolytic enzyme, pro-apoptotic molecule, or any molecule which modifies the target nucleic acid or target polypeptide, as disclosed above.

In some embodiments, The use of a cell death split biomolecular conjugate as disclosed herein is useful for the treatment of cancer, where the probes of cell death split-biomolecular conjugate recognize a particular target nucleic acid sequence or target polypeptide that are associated with a disorder such as cancer. For example, the probes of cell death split-biomolecular conjugate can recognize, but are not limited to HER2/Her-2, BRAC1 and BRAC2, Rb, p53 etc, as discussed above.

In some embodiments, a cancer, or disease or disorder or malignancy can be any disease of an organ or tissue in mammals characterized by poorly controlled or uncontrolled multiplication of normal or abnormal cells in that tissue and its effect on the body as a whole. In some embodiments, cancers comprise benign neoplasms, dysplasias, hyperplasias as well as neoplasms showing metastatic growth or any other transformations like e.g. leukoplakias which often precede a breakout of cancer. Cells and tissues are cancerous when they grow more rapidly than normal cells, displacing or spreading into the surrounding healthy tissue or any other tissues of the body described as metastatic growth, assume abnormal shapes and sizes, show changes in their nucleocytoplasmatic ratio, nuclear polychromasia, and finally may cease. Cancerous cells and tissues can affect the body as a whole when causing paraneoplastic syndromes or if cancer occurs within a vital organ or tissue, normal function can be impaired or halted, with possible fatal results. In some instances, if the function of a vital organ is compromised by cancer or cancer cells, either primary or metastatic, cancer can lead to the death of a subject or mammal affected. A malignant cancer is a cancer which has a tendency to spread and can cause death if not treated. Benign tumors usually do not cause death, although they can lead to death if they interfere with a normal body function by virtue of their location, size, or paraneoplastic side effects.

The term “cancer” as used herein refers to, but is not limited to, simple benign neoplasia but also comprises any other benign and malign neoplasia like 1) Carcinoma, 2) Sarcoma, 3) Carcinosarcoma, 4) Cancers of the blood-forming tissues, 5) tumors of nerve tissues including the brain, 6) cancer of skin cells. Cancer according to carcinoma, occurs in epithelial tissues, which cover the outer body (the skin) and line mucous membranes and the inner cavitary structures of organs e.g. such as the breast, lung, the respiratory and gastrointestinal tracts, the endocrine glands, and the genitourinary system. Ductal or glandular elements may persist in epithelial tumors, as in adenocarcinomas like e.g. thyroid adenocarcinoma, gastric adenocarcinoma, uterine adenocarcinoma. Cancers of the pavement-cell epithelium of the skin and of certain mucous membranes, such as e.g. cancers of the tongue, lip, larynx, urinary bladder, uterine cervix, or penis, may be termed epidermoid or squamous-cell carcinomas of the respective tissues and are in the scope of the definition of cancer as well. Cancer that are sarcoma develops in connective tissues, including fibrous tissues, adipose (fat) tissues, muscle, blood vessels, bone, and cartilage like e.g. Osteogenic sarcoma; liposarcoma, fibrosarcoma, synovial sarcoma. Cancer that are carcinosarcoma are cancers that develops in both epithelial and connective tissue. Cancer disease within the scope of this definition may be primary or secondary, whereby primary indicates that the cancer originated in the tissue where it is found rather than was established as a secondary site through metastasis—54 from another lesion.

Cancers and tumor diseases within the scope of this definition can be benign or malignant and can affect any and/or all anatomical structures of the body of a mammal. By example, but not limited to, cancers can comprise cancers and tumor diseases of (I) the bone marrow and bone marrow derived cells (leukemias), (II) the endocrine and exocrine glands like e.g. thyroid, parathyroid, pituitary, adrenal glands, salivary glands, pancreas, (III) the breast, like e.g. benign or malignant tumors in the mammary glands of either a male or a female, the mammary ducts, adenocarcinoma, medullary carcinoma, comedo carcinoma, Paget's disease of the nipple, inflammatory carcinoma of the young woman, (IV) the lung, (V) the stomach, (VI) the liver and spleen, (VII) the small intestine, (VIII) the colon, (IX) the bone and its supportive and connective tissues like malignant or benign bone tumor, e.g. malignant osteogenic sarcoma, benign osteoma, cartilage tumors; like malignant chondrosarcoma or benign chondroma; bone marrow tumors like malignant myeloma or benign eosinophilic granuloma, as well as metastatic tumors from bone tissues at other locations of the body; X) the mouth, throat, larynx, and the esophagus, XI) the urinary bladder and the internal and external organs and structures of the urogenital system of male and female like ovaries, uterus, cervix of the uterus, testes, and prostate gland, XII) the prostate, XIII) the pancreas, like ductal carcinoma of the pancreas; XIV) the lymphatic tissue like lymphomas and other tumors of lymphoid origin, XV) the skin, XVI) cancers and tumor diseases of all anatomical structures belonging to the respiration and respiratory systems including thoracic muscles and linings, XVII) primary or secondary cancer of the lymph nodes XVIII) the tongue and of the bony structures of the hard palate or sinuses, XVIV) the mouth, cheeks, neck and salivary glands, XX) the blood vessels including the heart and their linings, XXI) the smooth or skeletal muscles and their ligaments and linings, XXII) the peripheral, the autonomous, the central nervous system including the cerebellum, XXIII) the adipose tissue.

In some certain embodiments, a cancer is lymphoma; leukemia; sarcoma; adenomas. In some embodiments, a cancer is acute lympoblastic leukemia (ALL).

Another aspect of the present invention related to the use of a cell death split-biomolecular conjugate to treat a pathogen infection. In some embodiments, the method comprising contacting the pathogen infected cell with a cell death split-effector molecule; where the probes of the cell death split-effector polypeptide specifically recognize a particular target nucleic acid or target polypeptide that is present within the cell infected with a pathogen, and when the split-effector polypeptide fragments combine to form an active effector molecule in the presence of a particular target nucleic acid or target polypeptide that is associated with the pathogen infection.

In some embodiments, the cell death split biomolecular conjugate useful in the treatment of an infection with a pathogen comprises probes that recognize target nucleic acid sequences and/or target polypeptides specific to the pathogen, such as, for example virus coat proteins or viral genomic DNA.

In some embodiments, the split-biomolecular conjugates are useful for the treatment of pathogens such as, for example, but are not limited to; pathogens that potentially leading to infections and infectious diseases. Infections of the skin and underlying tissue are due to pathogens include, for example, cellulitis, necrotizing fasciitis, skin gangrene, lymphadenitis, acute lymphangitis, impetigo, skin abscesses, folliculitis, boils (furuncles), erysipelas, carbuncles (clusters of boils and skin abscesses), staphylococcal scalded skin syndrome, erythrasma or paronychia (can be caused by many bacteria and fungi). Most of these are bacterial infections. The most common bacterial skin infections are caused by Staphylococcus and Streptococcus. Skin infections caused by fungi are ringworm, a fungal skin infection caused by several different fungi and generally classified by its location on the body. Examples are Athlete's foot (foot ringworm, caused by either Trichophyton or Epidermophyton), jock itch (groin ringworm, can be caused by a variety of fungi and yeasts), scalp ringworm, caused by Trichophyton or Microsporum), nail ringworm and body ringworm (caused by Trichophyton). Candidiasis (yeast infection, moniliasis) is an infection by the yeast Candida. The following types of candida infections can be distinguished: Infections in skinfolds (intertriginous infections), vaginal and penile candida infections (vulvovaginitis), thrush, Perleche (candida infection at the corners of the mouth), candidal paronychia (candida growing in the nail beds, produces painful swelling and pus). Candida can also lead to generalized systemic infections especially in the immunocompromised host. Tinea versicolor is a fungal infection that causes white to light brown patches on the skin. The skin can also be affected by parasites, mainly tiny insects or worms. Examples are scabies (mite infestation), lice infestation (pediculosis, head lice and pubic lice are two different species), or creeping eruption (cutaneous larva migrans, a hookworm infection). Many types of viruses invade the skin. Examples are papillomavirusses (causing warts), herpes simplex virus (causing e.g. cold sores), or members of the poxvirus family (molluscum contagiosum (infection of the skin, causing skin-colored, smooth, waxy bumps).

In some embodiments, the split-biomolecular conjugates are useful for the treatment of pathogens such as, for example, are bacteria. Bacteremia refers to the presence of bacteria in the bloodstream, and where there are too many bacteria to be removed easily sepsis develops, causing severe symptoms. In some cases, sepsis leads to a life-threatening condition called septic shock. Bacilli are a type of bacteria classified according to their distinctive rod-like shape. Bacteria are either spherical (coccal), rod-like (bacillary), or spiral/helical (spirochetal) in shape. Gram-positive or gram-negative bacilli are distinguished Examples of gram-positive bacillary infections are are erysipelothricosis (caused by Erysipelothrix rhusiopathiae), listeriosis (caused by Listeria monocytogenes), and anthrax (caused by Bacillus anthracis). Within anthrax, pulmonary anthrax, gastrointestinal anthrax and anthrax skin sores can be distinguished. Examples of gram-negative bacillary infections are Hemophilus infections, Hemophilus influenzas infections, Hemophilus ducreyi (causes chancroid), Brucellosis (undulant, Malta, Mediterranean, or Gibraltar fever, caused by Brucella bacteria), tularemia (rabbit fever, deer fly fever, caused by Francisella tularensis), plague (black death, caused by Yersinia pestis, bubonic plaque, pneumonic plague, septicemic plague and pestis minor are distinguished), cat-scratch disease (caused by the bacterium Bartonella henselae), Pseudomonas infections (especially Pseudomonas aeruginosa), infections of the gastrointestinal tract or blood caused by Campylobacter bacteria (e.g. Campylobacter wlori [Helicobacter pylori]), cholera (infection of the small intestine caused by Vibrio cholerae), infections with other Vibrio spp., Enterobacteriaceae infections (cause e.g. infections of the gastrointestinal tract, members of the group are Salmonella, Shigella, Escherichia, Klebsiella, Enterobacter, Serratia, Proteus, Morganella, Providencia, and Yersinia), Klebsella pneumonia infections (severe lung infection), typhoid fever (caused by Salmonella typhi), nontyphoidal Salmonella infections, or Shigellosis (bacillary dysentery, an intestinal infection caused by Shigella bacteria). Bacteria that have a spherical shape are called cocci. Cocci that can cause infection in humans include staphylococci, streptococci (group A streptococci, group B streptococci, groups C and G streptococci, group D streptococci and enterocooci), pneumococci (cause e.g. pneumonia, thoracic empyema, bacterial meningitis, bacteremia, pneumococcal endocarditis, peritonitis, pneumococcal arthritis or otitis media), and meningococci. Toxic shock syndrome is an infection usually caused by staphylococci, which may rapidly worsen to severe, untreatable shock.

In some embodiments, the pathogen is Meningococci (Neisseria meningitidis) may cause infection of the layers covering the brain and spinal cord (meningitis). Neisseria gonorrhoeae cause gonorrhea, a sexually transmitted disease. Spirochetal Infections are infections with spirochetes, corkscrew-shaped bacteria. Examples include infections with Treponema, Borrelia, Leptospira, and Spirillum. Treponematoses (e.g. yaws, pinta) are caused by a spirochete that is indistinguishable from Treponema pallidum (causes syphilis). Relapsing fever (tick fever, recurrent fever, or famine fever) is a disease caused by several strains of Borrelia bacteria.

In another embodiment, a pathogen can be Lyme disease (transmitted by deer ticks) is caused by the spirochete Borrelia burgdorferi. Other examples for infections with spirochetes are Leptospirosis (a group of infections including Weil's syndrome, infectious (spirochetal) jaundice, and canicola fever), or rat-bite fever).

Disease-causing anaerobic bacteria include clostridia, peptococci, and peptostreptococci. Other examples are Bacteroides fragilis, Prevotella melaminogenica and Fusobacterium. Infections with anaerobic bacteria include dental abscesses, jawbone infections, periodontal disease, chronic sinusitis and middle ear infection, and abscesses in the brain, spinal cord, lung, abdominal cavity, liver, uterus, genitals, skin, and blood vessels. Examples for Clostridial infections tetanus (lockjaw, caused by the bacterium Clostridium tetani), or Actinomycosis (a chronic infection caused mainly by Actinomyces israelii).

In another embodiment, a pathogen can be Mycobacteria which causes Tuberculosis and leprosy, in particular by the airborne pathogen Mycobacterium tuberculosis, M. bovis, or M. africanum. Leprosy (Hansen's disease) is caused by the bacterium Mycobacterium leprae. Rickettsial infections are also known. Examples of diseases caused by Rickettsiae or Ehrlichieae are murine typhus (caused by Rickettsia typhi), Rocky Mountain spotted fever (caused by Rickettsia rickettsii), epidemic typhus (Rickettsia prowazekii), scrub typhus (Rickettsia—62 tsutsugamushi), Ehrlichiosis (Ehrlichia cants or closely related species), Rickettsial-pox, (Rickettsia akari), Q fever (Coxiella burnetii), or trench fever (Bartonella quintana).

In other embodiments, a pathogen can be a parasite such as a single-celled animal (protozoan) or worm, that survives by living inside another, usually much larger, organism. Examples for parasitic infections are—Amebiasis (caused by Entamoeba histolytica), Giardiasis (Giardia lamblia), Malaria (Plasmodium), Toxoplasmosis (Toxoplasma gondii), Babesiosis (Babesia parasites), Trichuriasis (Trichuris trichiura, an intestinal roundworm), Ascariasis (Ascaris lumbricoides), Hookworm Infection (Ancylostoma duodenale or Necator americanus), Trichinosis (Trichinella spiralis), Toxocariasis (visceral larva migrans, caused by the invasion of organs by roundworm larvae, such as Toxocara cants and Toxocara cat)), Pork tapeworm infection (Taenia solium), or Fish tapeworm infection (Diphyllobothrium latum).

In another embodiment, a pathogen can be a fungus. Fungi tend to cause infections in people with a compromised immune system. Examples for fungal infections are Histoplasmosis (caused by Histoplasma capsulatum), Coccidioidomycosis (Coccidioides immitis), Blastomycosis (Blastomyces dermatitidis), Candidiasis (caused by strains of Candida, especially Candida albicans), or Sporotrichosis (Sporothrix schenckii).

In another embodiment, the pathogen can be a virus. Non-limiting examples of viral infections are as follows; Respiratory viral infections are, for example, common cold (caused by Picornaviruses [e.g. rhinoviruses], Influenza viruses or respiratory syncytial viruses), Influenza (caused by influenza A or influenza B virus), Herpesvirus Infections (herpes simplex, herpes zoster, Epstein-Barr virus, cytomegalovirus, herpesvirus 6, human herpesvirus 7, or herpesvirus 8 (cause of Kaposi's sarcoma in people with AIDS), central nervous system viral infections (e.g. Rabies, Creutzfeldt-Jakob disease (subacute spongiform encephalopathy), progressive multifocal leukoencephalopathy (rare manifestation of polyomavirus infection of the brain caused by the JC virus), Tropical spastic paraparesis (HTLV-I), Arbovirus infections (e.g. Arbovirus encephalitis, yellow fever, or dengue fever), Arenavirus Infections (e.g. Lymphocytic choriomeningitis), hemorrhagic fevers (e.g. Bolivian and Argentinean hemorrhagic fever and Lassa fever, Hantavirus infection, Ebola and Marburg viruses).

One example of a common virus is Human immunodeficiency virus (HIV) infection is an infection caused by HIV-1 or HIV-II virus, which results in progressive destruction of lymphocytes. This leads to acquired immunodefciency syndrome (AIDS). Other viruses include for example Hepatitis A, hepatitis B, hepatitis C, SARS, avian flu etc.

Other pathogen viruses include sexually transmitted (venereal) diseases, for example syphilis (caused by Treponema pallidum), gonorrhea (Neisseria gonorrhoeae), ehaneroid (Hemophilus duereyi), lymphogranuloma venereum (Chlamydia traehomatis), granuloma inguinale (Calymmatobaeterium granulomatis), nongonoeoeeal urethritis and ehlamydial eervieitis (caused by Chlamydia traehomatis, Ureaplasma urealytieum, Triehomonas vaginalis or herpes simplex virus), triehomoniasis (Triehomonas vaginalis), genital candidiasis, genital herpes, genital warts (caused by papillomaviruses), or HIV infection.

In another embodiment, a pathogen can be an infection with opportunistic pathogens, often infecting people with impaired immune system, such as for example but are not limited to nocardiosis (caused by Nocardia asteroides), aspergillosis, mucormyeosis, and eytomegalovirus infection.

In some embodiments, a cell death split biomolecular conjugates as disclosed herein can be used in the treatment of cells other than tumor cells or virus infected cells. For example, in some embodiments, the split-biomolecular conjugate that carries a cytotoxic effector molecule can specifically target cells for example B cells, which secrete antibodies directed against itself. In some embodiments, the biomolecular conjugates as disclosed herein are useful in the treatment of autoimmune, or autoimmune-related diseases, for example but not limited to; Hashimoto's thyroiditis; pernicious anemia; Addison's disease; type I diabetes; rheumatoid arthritis; systemic lupus erythematosus; dermatomyositis; Sjogren's syndrome; lupus erythematosus; multiple sclerosis; myasthenia gravis; Reiter's syndrome; and Grave's disease.

As used herein, the term “autoimmune disease” or autoimmune-related disease refers to illnesses or diseases which occur when the bodies tissues are attacked by its own immune system. The immune system is a complex organization within the body that is designed normally to “seek and destroy” invaders of the body, including infectious agents. Patients with autoimmune diseases frequently have unusual antibodies circulating in their blood that target their own body tissues. Examples of autoimmune diseases include systemic lupus erythematosus, Sjogren syndrome, Hashimoto thyroiditis, rheumatoid arthritis, juvenile (type 1) diabetes, polymyositis, scleroderma, Addison disease, vitiligo, pernicious anemia, glomerulonephritis, multiple sclerosis, and pulmonary fibrosis.

In another embodiment, the split-biomolecular conjugate as disclosed herein can be used to sensitize the cell to a second agent, so that the second agent will specifically affect only those cells that are sensitized. Typically the second agent is a chemical or physical entity or agent that triggers cell death of a targeted cell. In such an embodiment, the “sensitizing split-biomolecular conjugate” comprises a split-effector molecule which reassemble to form an active effector molecule in the presence of the target nucleic acid sequence or target polypeptide that is capable of sensitizing the cell to other secondary agents. In some embodiments, a examples of effector molecules include, for example but not limited to β glucuronidase enzymes; hypoxanthine-guanine phosphoribosyltransferase, β-lactamase enzymes and carboxylesterase HCE1, as discussed above. In some embodiments, such split-biomolecular conjugates are referred to herein as “sensitizing split-biomolecular conjugates” and in some embodiments, they are useful for the treatment of cancers and/or pathogen infections, where a target nucleic acid sequence or target polypeptide contributes to a symptom of the cancer or pathogen infection respectively.

In another embodiment, a biomolecular conjugate as disclosed herein can be used to trigger the degradation or destruction of a target nucleic acid sequence and/or target polypeptide, thereby either killing the cell or eliminating the pathogenic target nucleic acid and/or polypeptide from the cell. In such an embodiment, the split-biomolecular conjugate is referred to as a “degrading split-biomolecular conjugate” comprises a split-effector molecule which, when in the active effector configuration in the presence of the target nucleic acid or polypeptide, functions as a nuclease or protease or triggers nucleic acid degradation or protein degradation of the target nucleic acid or target polypeptide in the cell. In such an embodiment, the effector molecule can be, for example, but not limited to nucleases; proteases and ubiquitinases. In some embodiments, degrading split-biomolecular conjugates can be used to treat cancers (such as those disclosed herein) and pathogen infections (such as those disclosed herein) and other diseases and disorders due to the presence of a pathogenic nucleic acid or pathogenic peptide.

In some embodiments, a disease, disorder or malignancy refers to any disease, disorder or malignancy where a symptom is caused, in part or wholly by a pathological nucleic acid sequence or pathological polypeptide. For example, a neural disease which affect the nervous system, respiratory diseases, cardiovascular disorders, hepatic disorders; inflammatory diseases; pancreatic diseases, digestive organ diseases, renal diseases, skin diseases; lung diseases etc.

In some embodiments, neural diseases and neurodegenerative diseases are, for example but not limited to, cerebral infarction, cerebrovascular accidents, Parkinson's disease, Alzheimer disease, Huntington's chorea, spinal cord injury, depression, manic-depression psychosis, amyotropic lateral sclerosis (ALS), and other neurodegenerative diseases and the like. In some embodiments, respiratory organ system diseases include chronic obstructive lung disease, pulmonary emphysema, bronchitis, asthma, interstitial pneumonia, pulmonary fibrosis and the like.

In some embodiments, cardiovascular disorders are, for example but not limited to, obstructive vascular disease, myocardial infarction, cardiac failure, coronary artery disease and the like. As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. In some embodiments, hepatic diseases include hepatitis B, hepatitis C, alcoholic hepatitis, hepatic cirrhosis, hepatic insufficiency and the like, and pancreatic diseases include diabetes mellitus, pancreatitis and the like. The digestive organ system diseases include Crohn disease, ulcerative colitis and the like. Renal diseases include IgA glomerulonephritis, glomerulonephritis, renal insufficiency and the like, and skin diseases include decubitus, burn, sutural wound, laceration, incised wound, bite wound, dermatitis, cicatricial keloid, keloid, diabetic ulcer, arterial ulcer, venous ulcer and the like. Lung diseases include emphysema, chronic bronchitis, chronic obstructive lung disease, cystic fibrosis, idiopathic interstitial pneumonia (pulmonary fibrosis), diffuse pulmonary fibrosis, tuberculosis, asthma and the like.

As used herein, inflammatory diseases refer to diseases triggered by cellular or non-cellular mediators of the immune system or tissues causing the inflammation of body tissues and subsequently producing an acute or chronic inflammatory condition. Examples of such inflammatory diseases include, but are not limited to, hypersensitivity reactions of type I-IV, for example but not limited to hypersensitivity diseases of the lung including asthma, atopic diseases, allergic rhinitis or conjunctivitis, angioedema of the lids, hereditary angioedema, antireceptor hypersensitivity reactions and autoimmune diseases, Hashimoto's thyroiditis, systemic lupus erythematosus, Goodpasture's syndrome, pemphigus, myasthenia gravis, Grave's and Raynaud's disease, type B insulin-resistant diabetes, rheumatoid arthritis, psoriasis, Crohn's disease, scleroderma, mixed connective tissue disease, polymyositis, sarcoidosis, glomerulonephritis, acute or chronic host versus graft reactions.

In another embodiment, the split-biomolecular conjugate as disclosed herein can be used to trigger the survival or the cell, for example inhibiting the cell death pathway and/or activating the cell survival pathway. In such an embodiment, the split-biomolecular conjugate is referred to as a “survival split-biomolecular conjugate” and can comprise a split-effector molecule, which in the presence of a particular target nucleic acid or target polypeptide that is capable of initiating a cell survival pathway or inhibiting cell death in the cell. In some embodiments, an effector molecule can be, for example, but not limited to, anti-apoptotic molecules such as bcl-2, hsp70, hsp27, IAP proteins etc. In such an embodiment, survival split-biomolecular conjugates can be used to treat diseases and disorders, including pathogen infections which result in the selective loss of cells as due to the presence of a pathogenic nucleic acid or pathogenic peptide. For example, such diseases include but are not limited to all degenerative diseases, such as neurodegenerative diseases, for example, Parkinson's disease, Alzheimer disease, Huntington's chorea, spinal cord injury, amyotropic lateral sclerosis (ALS), and muscular disorders such as muscular dystrophy etc.

In another embodiment, a split-biomolecular conjugate can be used to selectively replace a lost or reduced expression or dysfunctional polypeptide in the cell, where the effector molecule functions as the replacement polypeptide. In such an embodiment, such a split biomolecular conjugate is referred to as a “proxy split-biomolecular conjugate” and can comprise a split-effector molecule which reassembles to form a replacement polypeptide in the presence of a particular target nucleic acid or target polypeptide that is capable of replacing a dysfunctional or lost polypeptide in the cell. In some embodiments, a proxy split-biomolecular conjugate can be used to treat any disease or disorder where a symptom of the disease is due to the loss of, reduced expression or a polypeptide, or expression of a dysfunctional or mutated polypeptide that contributes to the pathogenesis of the diseases or disorder. Examples of such diseases include, but are not limited to, loss of function diseases such as muscular dystrophy which has loss of the protein dysferlin, cystic fibrosis etc. In some embodiments, the disease can be the result of, for example, a genetic predisposition to a disease or an acquired disease.

In some embodiments, the present invention relates to a method of selectively killing cells or keeping cells alive, or assessing the characteristics of a new polypeptide or the effect of degrading a target nucleic acid or polypeptide using a selective split-biomolecular conjugates as disclosed herein. In some embodiments, a cell-death split-biomolecular conjugate and/or survival split-biomolecular conjugates can be used to kill or promote the survival of selective cells respectively. In some embodiments, the methods can be used for cell separation in vitro by selectively killing unwanted types of cells, for example, by selectively killing or keeping selected cells alive in a population of cells in bone marrow prior to transplantation into a patient undergoing marrow ablation by radiation.

In all of the above embodiments, the subject to be treated is a mammal, including humans and non-human mammals and animals in general, for example, mammals, non-human animals such as farm animals comprising, but not limited to: cattle, horses; goats; sheep; pigs; donkeys; etc. household pets including, but not limited to: cats; dogs; rodents comprising but not limited to: rabbits, mice; hamsters; etc; birds and poultry and other livestock and fowl.

### Methods for Generation of Split-Biomolecular Conjugates and Assessment of Target-Mediated Protein Complementation of Effector Molecules

Another aspect of the present invention relates to the generation of the split-biomolecular conjugates as disclosed herein. In some embodiments, the method comprises assessing the protein structure of the effector molecules and determining appropriate sites for splitting the effector molecule. The method further comprises expressing the protein fragments and assessing their complementation ability in the presence and absence of conjugated probes, further in the presence and absence of target polypeptides or target nucleic acid sequences.

### (i) Design of Locations of Split Sites in Effector Polypeptide

Optimal splitting can be determined by assessing structural conformation and by assessing alternative splitting points. In some embodiments, several cloning attempts may be required to achieve complementary split-effector fragments that do not result in spontaneous reassembly, or to obtain two split-effector fragments that can reassemble efficiently to form an active effector protein, especially when mediated by the attached probes recognizing a target nucleic acid sequence or target polypeptide.

Of note, the methods as disclosed herein to design split sites in the effector polypeptides can also be used to identify split sites in polypeptide probe proteins to generate fragments which can be used as complementary partners of a split-polypeptide probe.

In some embodiments, the following criteria can be followed in choosing the three split points for initial testing of splitting an effector protein into two or more split-effector protein fragments: 1) split point should separate the activity-important amino acids between the two protein halves; 2) split point should be located within the unstructured region (to introduce a minimal structural disturbance to the split protein halves); 3) split protein halves correspond to compact folding unit within the full-size effector molecule such as RTA.

In some embodiments, a further criteria can be applied, for example, fragments of split effector proteins should not have large hydrophobic surfaces making proteins aggregation-prone. Methods to identify surface hydrophobicity of proteins is known in the art, for example though protein solubility prediction software, for example http://www.biotech.ou.edu.

Of note, due to the chance of complementary split-effector protein fragments forming inclusion bodies, it is important to test multiple different split sites in a effector molecule, for example at least 5, at least 6, at least 7, at least 8, at least 9 at least 10 or more than 10 different split sites in a single effector molecule. Each combination of complementary split-effector protein fragments should be tested for efficiency of expression with minimal formation of inclusion bodies and then for efficiency of protein complementation in the presence or absence of a target as disclosed herein.

(ii) Expressing Split-Effector Protein Fragments with Minimal Formation of Inclusion Bodies.

Production of the split-biomolecular conjugates, or split-effector fragments can be performed using in vitro expression systems commonly known by persons of ordinary skill in the art. Proper protein refolding of expressed effector fragments is important to ensure they are able to re-assemble to form a functionally active effector protein. In some embodiments, such expression systems include, for example systems which limit the production of inclusion bodies, for example but are not limited to methods as disclosed in EP1516928 and US20050130259, and WO0039310 which are specifically incorporated herein by reference.) Cell-free gene/protein expression

Cell Free Expression of the Split-Effector Protein Fragments

In some embodiments, cell-free gene expression is useful to express the split-effector biomolecular conjugates. In some embodiments the nucleic acid encoding the split-effector protein is transcribed in vitro by an RNA polymerase, e.g. T7 RNA polymerase, and then the RNA is subsequently translated using a cellular lysate, e.g. obtained from E. coli. Cell-free protein expression systems, for example rapid translation systems (RTS) are commonly known by persons of ordinary skill in the art, and are commercially available, for example from Roche Applied Science¹ or Novagen² as the coupled transcription/translation kits. In some embodiment, use of such kits are capable to generate micrograms to milligrams of desired protein within several hours from the PCR-generated linear DNA templates containing all necessary regulatory elements (promoters, terminators, etc) and tag sequences for subsequent purification.

Using such methods, the split-effector protein fragments, such as split-RTA fragments can be obtained rapidly and quickly with minimal in vivo cloning, therefore allowing more readily obtain the range of RTA fragments corresponding to various split points.

Alternatively, in some embodiments, cell-free gene expression systems are useful to produce the split-effector protein fragments as disclosed herein to produce the proteins in soluble form. Such systems are useful to as the reduced macromolecular crowding inside a RTS reaction chamber is beneficial and promotes for correct protein folding, thus reducing the formation of incorrectly folded insoluble inclusion bodies.

In some embodiments, where the cell-free expressed split-effector protein fragment is still poor soluble, solubility-enhancing additives can easily be included and/or certain solubility-promoting fusion tags, for example, such as but not limited to MBP, Trx or Nus sequences, can be added to the expressed insoluble protein by overlap extension PCR to make this protein(s) soluble. In some embodiments, some potential problems with cell-free expression could occur due to a possibly tight mRNA structure decreasing the expression efficiency in vitro as well as solubility tags might interfere with proper reassembly of split protein toxin. Therefore, to effectively express split-effector protein fragments, multiple cell-free expression systems can be utilized, as well as bacterial and insect expression systems.

2) Cell-Based Gene Expression of the Split-Effector Protein Fragments

In some embodiment, effector fragments can be expressed in an in vitro expression system and secreted into the soluble cellular fraction of the cells and harvested from the supernatant or medium surrounding the cells.

In some embodiments, one method for expressing a split-effector protein fragment useful in the methods as disclosed herein, is using specific bacterial host strains, such as E. coli strains that excrete overexpressed proteins out of cells, thus minimizing formation of intracellular inclusion bodies. In some embodiments, host bacterial cells, such as E. coli cells produce the bacteriocin release protein (BRP)³, which facilitates secretion of intracellular proteins into the culture medium, where they can undergo correct protein folding. Accordingly, use of such expression systems are useful for the expression of split-effector protein fragments with minimal chance of formation of incorrectly folded insoluble inclusion bodies.

In further embodiments, the present invention relates to assessing the formation of an active effector proteins by protein complementation of the split-effector fragments. For example, the expressed split-effector protein fragments can be conjugated to a probe, and assessment of the function of the effector protein to identify fragments that spontaneously protein complement in the absence of a target. Such split-effector fragments which spontaneously complement in the absence of a target to the conjugated probe can be discarded, as these indicate non-specific protein complementation of the split-biomolecular conjugate in the absence of a target.

If reassembly of the complementing split-effector fragments does occur spontaneously, i.e. in the absence of the target nucleic acid sequence or target polypeptide, one can modify the effector fragments to introduce mutations which prevent self-assembly but does not alter the function or activity of the effector protein when the two fragments are associated by protein complementation. The effect of an introduced modification or mutation on effector protein activity can be compared with the activity of an intact effector molecule (i.e. an effector molecule which has not been split into two complementary fragments)

Split-effector protein fragment would and the proteins which do not spontaneously protein complement in the absence of a target can be selected for further analysis. Such split-effector protein fragments can be further analyzed and to identify the split-effector protein fragments that complement only in the presence of a target molecule, such as a nucleic acid target or protein target to the probe which is conjugated to the split-effector protein fragments.

(iii) Assessing Target-Mediated Protein Complementation of Split-Effector Protein Fragments.

As discussed above, the efficiency of protein complementation of a two complementary split-effector protein fragments in the absence and presence of a probe is assessed. In some embodiments, the formation of the split-biomolecular protein conjugate fragments can occur by conjugation of the split-effector protein fragments with a probe, for example a nucleic acid probe or a polypeptide probe.

Conjunction Methods: a Variety of Conjugation Methods can be Used

The term “conjugate” or “conjugated” refer to the joining of two or more entities. The joining can be fusion of the two or more polypeptides, or covalent, ionic, or hydrophobic interactions whereby the moieties of a molecule are held together and preserved in proximity. The attachment of the entities may be together by linkers, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat sensitive linkers.

The attachment can be by means of linkers, chemical modification, peptide linkers, chemical linkers, covalent or non-covalent bonds, or protein fusion or by any means known to one skilled in the art. The joining can be permanent or reversible. In some embodiments, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention.

According to the present invention, the split-effector protein fragment, can be linked to the probe via any suitable means, as known in the art, see for example U.S. Pat. Nos. 4,625,014, 5,057,301 and 5,514,363, which are incorporated herein in their entirety by reference. For example, the split-effector protein fragment can be covalently conjugated to the probe, either directly or through one or more linkers. In one embodiment, the split-effector protein fragment of the present invention is conjugated directly to probe. In another embodiment, the split-effector protein fragment of the present invention is conjugated to a probe via a linker, e.g. a transport enhancing linker.

A large variety of methods for conjugation of split-effector protein fragments with probes are known in the art. Such methods are for e.g. described by Hermanson (1996, Bioconjugate Techniques, Academic Press), in U.S. Pat. No. 6,180,084 and U.S. Pat. No. 6,264,914 which are incorporated herein in their entirety by reference and include e.g. methods used to link haptens to carriers proteins as routinely used in applied immunology (see Harlow and Lane, 1988, “Antibodies: A laboratory manual”, Cold Spring Harbor Laboratory Press, Cold Spring Harbor, N.Y.). It is recognized that, in some cases, a split-effector protein fragment can lose efficacy or functionality upon conjugation depending, e.g., on the conjugation procedure or the chemical group utilized therein. However, given the large variety of methods for conjugation the skilled person is able to find a conjugation method that does not or least affects the efficacy or functionality of the entities to be conjugated.

Suitable methods for conjugation of a split-effector protein fragments with probe include e.g. carbodimide conjugation (Bauminger and Wilchek, 1980, Meth. Enzymol. 70: 151-159). Alternatively, a moiety can be coupled to a targeting agent as described by Nagy et al., Proc. Natl. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde cross-linking.

One can use a variety of different linkers to conjugate split-effector protein fragments as described herein to a probe such as a nucleic acid probe, for example but not limited to aminocaproic horse radish peroxidase (HRP) or a heterobiofunctional cross-linker, e.g. carbonyl reactive and sulfhydryl-reactive cross-linker. Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multistep protocols can offer a great control of conjugate size and the molar ratio of components.

The term “linker” refers to any means to join two or more entities, for example a peptide with another peptide, or a liposome. A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling. It will be appreciated that modification which do not significantly decrease the function of split-effector protein fragments, and/or the probe are preferred.

### Method for Screening a Pathogenic Target Nucleic Acid or Polypeptide in a Subject.

In some embodiments, the present invention provides a method to measure the level of a pathogenic target nucleic acid or pathogenic polypeptide in a subject comprising; administering to the subject an effective amount of a pharmaceutical composition of the split biomolecular conjugate comprising a split-detector molecule, wherein each of the split-detector polypeptide fragments are conjugated to at least one of two probes specific for a particular target nucleic acid or target polypeptide that is associated with a disease or disorder; formation of an active detector molecule, wherein the formation of an active effector molecule is facilitated by binding of at least two probes with the target nucleic acid or target polypeptide that is associated with a disease or disorder; measuring the level of the active detector molecule; wherein the level of the active detector molecule is a measure of the target nucleic acid or pathogenic polypeptide in a subject.

In some embodiments, a detector polypeptide is selected from a group comprising; β-lactamase; DFHR; luciferase; fluorescent protein or variants or fragments thereof.

In some embodiments, the level of the active detector molecule can be used to determine the level of pathogenic target nucleic acid or pathogenic polypeptide in a subject, for example, by measuring the level of a pathogenic target nucleic acid or pathogenic polypeptide using the methods as disclosed herein at a first timepoint, and comparing the level from the first timepoint, with the level of a pathogenic target nucleic acid or pathogenic polypeptide at a second time point. Such an embodiment is useful for determining the effectiveness of a treatment, for example a treatment of a subject with a split-biomolecular conjugate by the methods as disclosed herein. Accordingly, in some embodiments, a subject can be administered both a split-biomolecular conjugate comprising an effector molecule and a split-bimolecular conjugate comprising a detector molecule. In some embodiments, a split-biomolecular conjugate comprising an effector molecule can be administered simultaneously with a split-bimolecular conjugate comprising a detector molecule, or in alternative embodiments, they can administered sequentially, in any order and any number of times.

In some embodiments, a detector molecule is a fluorescent protein, for example, but not limited to, green fluorescent protein (GFP), enhanced green fluorescent protein (EGFP), green-fluorescent-like proteins; yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP) or a red fluorescent protein (dsRED), where one of the fragments in the reconstituted fluorescent protein contains a mature preformed chromophores. All of the above mentioned fluorescent proteins and fragments thereof that will result in a fluorescing fluorescent protein are encompassed for use in the present invention. Also encompassed are those fluorescent proteins known to those of skill in the art, and fragments and genetically engineered proteins thereof.

In some embodiments, the presence of a active detector protein, for example an active fluorescent protein is detectable by flow cytometry, fluorescence plate reader, fluorometer, microscopy, fluorescence resonance energy transfer (FRET), by the naked eye or by other methods known to persons skilled in the art. In an alternative embodiment, fluorescence is detected by flow cytometry using a florescence activated cell sorter (FACS) or time lapse microscopy.

In another embodiment of the invention, a detector molecule is an enzyme, such that when the split-detector protein fragments associated in close proximity to form an assembled, active enzyme, which can be detected using an enzyme activity assay. Preferably, the enzyme activity is detected by a chromogenic or fluorogenic reaction. In one preferred embodiment, the enzyme is dihydrofolate reductase (DHFR) or β-lactamase.

In another embodiment, the enzyme is dihydrofolate reductase (DHFR). For example, Michnick et al. have developed a “protein complementation assay” consisting of N- and C-terminal fragments of DHFR, which lack any enzymatic activity alone, but form a functional enzyme when brought into close proximity. See e.g. U.S. Pat. Nos. 6,428,951, 6,294,330, and 6,270,964, which are hereby incorporated by reference. Methods to detect DHFR activity, including chromogenic and fluoregenic methods, are well known in the art.

In alternative embodiments, other detector molecules can be used, for example, enzymes that catalyze the conversion of a substrate to a detectable product. Several such systems for split-polypeptide reassemblies include, but are not limited to reassembly of; β-galactosidase (Rossi et al, 1997, PNAS, 94; 8405-8410); dihyrofolate reductase (DHFR) (Pelletier et al, PNAS, 1998; 95; 12141-12146); TEM-1 β-lactamase (LAC) (Galarneau at al, Nat. Biotech. 2002; 20; 619-622) and firefly luciferase (Ray et al, PNAS, 2002, 99; 3105-3110 and Paulmurugan et al, 2002; PNAS, 99; 15608-15613). For example, split β-lactamase has been used for the detection of double stranded DNA (see Ooi et al, Biochemistry, 2006; 45; 3620-3525). Encompassed for use in the present invention are the use of activated split polypeptide fragments for real-time signal detection, wherein the fragments are in a fully folded mature conformation enabling rapid signal detection upon complementation.

### Pharmaceutical Composition

The present invention also relates to a pharmaceutical composition comprising split-biomolecular conjugates of the present invention in a pharmaceutically acceptable carrier. In therapeutic applications, compositions are administered to a patient suffering from a disease, in an amount sufficient to ameliorate or at least partially arrest the disease and its complications. An amount adequate to accomplish this is defined as a therapeutically effective dose. Amounts effective for this use will depend on the severity of the disease and the general state of the patient's health.

In one embodiment, the cells are treated with the split-biomolecular conjugate in vivo. In another embodiment, the cells are treated with the split-biomolecular conjugate ex vivo, where the cells are obtained from the subject and administered the pharmaceutical composition ex vivo, and in certain embodiments they are transplanted back into the subject.

In most embodiments, the subject treated with pharmaceutical composition is a mammal, including humans and non-human mammals and animals in general, for example, mammals, non-human animals such as farm animals comprising, but not limited to: cattle, horses; goats; sheep; pigs; donkeys; etc. household pets including, but not limited to: cats; dogs; rodents comprising but not limited to: rabbits, mice; hamsters; etc; birds and poultry and other livestock and fowl

Advantageously, the pharmaceutical composition is suitable for parenteral administration. The split biomolecular conjugates of the present invention may be administered by various means appropriate for different purposes, for example, for treating tumors in various parts of the body, according to methods known in the art for other similar compositions, such as immunotoxins (See, for example, Rybak, et al., Human Cancer Immunology, in IMMUNOLOGY AND ALLERGY CLINICS OF AMERICA, W. B. Saunders, 1990, and references cited therein). Accordingly, the present invention also relates to pharmaceutical compositions comprising split biomolecular conjugates of this invention and a pharmaceutically acceptable carrier, particularly such compositions which are suitable for the above means of administration.

Single or multiple administrations of the compositions may be administered depending on the dosage and frequency as required and tolerated by the patient. In any event, the composition should provide a sufficient quantity of the proteins of this invention to effectively treat the patient.

Preferably, the compositions for administration will commonly comprise preloaded polymetric nanoparticles and/or cataionic liposomes (Pattrick et al, 2001; Richardson et al., 2001; Sachdeva, 1998) comprising the split-biomolecular conjugate(s) in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.01 to 100 mg per patient per day. Dosages from 0.1 up to about 1000 mg per patient per day may be used, particularly when the drug is administered to a secluded site and not into the blood stream, such as into a tumor or an organ within which a tumor resides. Actual methods for preparing parenterally administrable compositions will be known or apparent to those skilled in the art and are described in more detail in such publications as REMINGTON'S PHARMACEUTICAL SCIENCE, 15TH ED., Mack Publishing Co., Easton, Pa., (1980).

The pharmaceutical composition can be administered by any means known to persons skilled in the art. For example, some methods include pump, direct injection, topical application, or administration to a subject via intrademal, subcutaneous, intravenous, intralymphatic, intranodal, intramucosal or intramuscular administration.

The present invention also relates the use of a pharmaceutical composition of a split-biomolecular conjugate of the invention in the preparation of a drug useful in the treatment of cancer or a viral disease or any other disease identified by persons skilled in the art whereby the methods in this invention could be used.

In one embodiment of the invention the split-biomolecular conjugates are expressed by means of inclusion bodies. “Inclusion bodies” (IBs), as used herein, refer to an insoluble form of polypeptides recombinantly produced after overexpression of the encoding nucleic acid in microorganisms/prokaryotes. There exist a large number of publications which describe the recombinant production of proteins in microorganisms/prokaryotes via the inclusion bodies route, and are any such method can be used for production of the split-biomolecular conjugates by persons skilled in the art. Examples of such reviews are Misawa, S., et al., Biopolymers 51 (1999) 297-307; Lilie, H., Curr. Opin. Biotechnol. 9 (1998) 497-501; Hockney, R. C., Trends Biotechnol. 12 (1994) 456-463.

In another embodiment, the biomolecular conjugates are produced within the cell by expression from an expression vector. Methods to introduce the vector into the cell are well known by persons skilled in the art and are encompassed for use in this invention, and include viral mediated mechanisms, naked DNA mechanisms, direct DNA injection etc.

The pharmacological compositions according to the invention may be used in conjunction with other treatments, for example if the split-biomolecular conjugate is used for the treatment of cancer, the pharmaceutical composition may be administered for example with any other anti-cancer therapy, chemotherapy and/or with anti-angliogenic treatment. If the split-biomolecular conjugate is used for the treatment of a pathogen, the pharmaceutical composition may be administered for example with one or more other anti-viral agents etc.

For further elaboration of general techniques useful in the practice of this invention, the practitioner can refer to standard textbooks and reviews in cell biology, tissue culture. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

As an example to demonstrate the production and functionality of a split-biomolecular conjugate, ricin A was used as the effector molecule to selectively target and kill Acute lymphoblasic Leukemia (ALL) cells, in particular Pediatric acute lumphoblastic leukemia (ALL) cells. This example is an example of the methods and production of a split biomolecular conjugate, and is not intended to limit the scope of the invention.

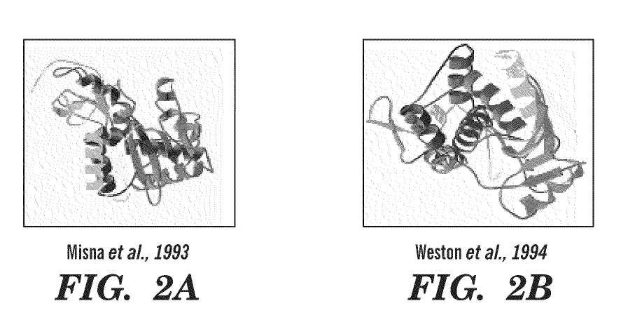

Ricin A-chain toxin (RTA) is highly efficient cytotoxic enzyme that destroys ribosomes (ribotoxin) and rapidly kills targeted cells (Hartley & Lord, 2004; Bigalke & Rummel, 2005). RTA is a 267-amino acid globular protein, and it has the three-domain structure (FIG. 2), with an arrangement of domains resembling a three-layer sandwich (Weston et al., 1994; Bigalke & Rummel, 2005). Domain I (˜120 amino acids) is formed by several β-sheets. It is connected by a loop to domain II formed by a few a-helixes, and the nuclease active site is formed by these two domains as a cleft in the middle of the RTA globule. Domain III plays a role of an interface to ricin B-chain (RTB), which is not necessary for toxicity. Thus, it is likely that functional RTA can be reassembles from two inactive protein fragments corresponding to domains I and II [or domains I and (II and III)]. Importantly, RTA can be obtained as a recombinant protein in E. coli (Weston et al, 1994). Also important is that the RTA re-assembly is supported by ribosomes (Argent et al, 1994); in vivo, RTA enters the cytosol as a partially unfolded protein that is then refolded by ribosomes.

RTA toxin has already been used in therapeutic studies (Lord et al, 1994, and www.ansci.comell.edu/plants/toxicagents/ricin/ricin.html#ricmech). It can be targeted to specific cancer cells, by conjugating the RTA chain to antibodies or growth factors that preferentially bind unwanted cells. These immunotoxins have worked very well in vitro applications, e.g. bone marrow transplants. Although they have not worked very well in many in vivo situations, progress in this area of research shows promise for the future. Importantly, RTA cannot enter the cells by itself (without ricin B chain) so that we do not expect significant toxic effect of RTA when, after destroying the cancer cells, it may re-enter the bloodstream. To be completely safe, free extracellular RTA can be blocked in our approach by the injection of corresponding antibodies (Mantis et al, 2006; Wang et al, 2006.) In bone marrow transplant procedures, RTA-immunotoxins have been used successfully to destroy T lympocytes in bone marrow taken from histocompatible donors. This reduces rejection of the donor bone marrow, a problem called “graft versus hosts disease” (GVHD). In steroid-resistant, acute GVHD situations, RTA-immunotoxins helped alleviate the condition. Also, in autologous bone marrow transplantation, a sample of the patient's own bone marrow is treated with anti-T cell immunotoxins to destroy malignant T-cells in T cell leukemias and lymphomas. Thus, ricin can be used for therapeutic applications of the protein complementation approach. Following the in vitro experiments, which show that there is no spontaneous reassembly of the split ricin A fragments, there is a great chance that nucleic acid dependent ricin-A reassembly can be adopted for targeted killing of cancer cells.

Acute lymphoblastic Leukemia. Pediatric acute lumphoblastic leukemia (ALL) is a heterogenous disease comprising different immunophenotypes and various genetic subtypes caused by chromosomal translocations with aberrant gene fusions that result in the expression of oncogenes. One of the most often translocation responsible for childhood ALL is t(12;21).

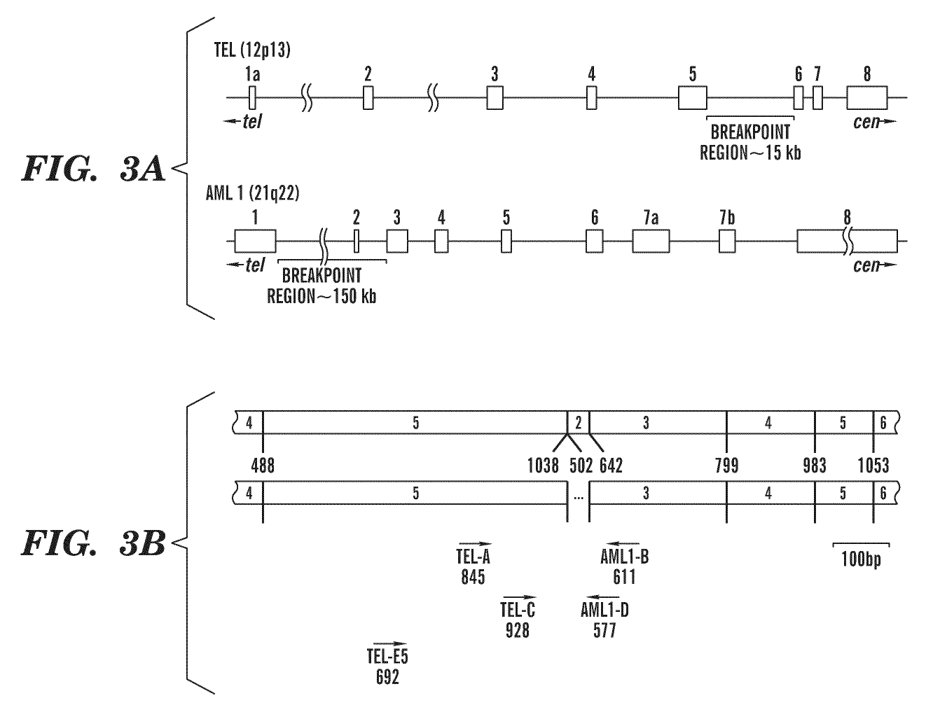

The TEL-AML1 fusion gene. The t(12;21) translocation creates a gene fusion that includes the 5′ portion of TEL, a member of the ETS family of transcription factor genes, and almost the entire coding region of another transcription factor gene, AML1, which encodes the α subunit of core binding factor, a master regulator of the formation of a definitive hematopoeitic stem cells (FIG. 3). The chimeric TEL-AML1 transcription factor retains an essential protein-protein interaction domain of TEL and the DNA-binding domain and translational regulatory sequences of AML1. A prominent effect of the TEL-AML1 fusion protein is inhibition of the transcriptional activity that is normally initiated when AML1 binds to a DNA region termed the core enhancer sequence. The abnormal TEL-AML1 fusion protein can bind to the core enhanced sequence but instead of activating transcription, it recruits histone deacetylases, which induce closure of the chromatin structure and, hence, inhibition of transcription.

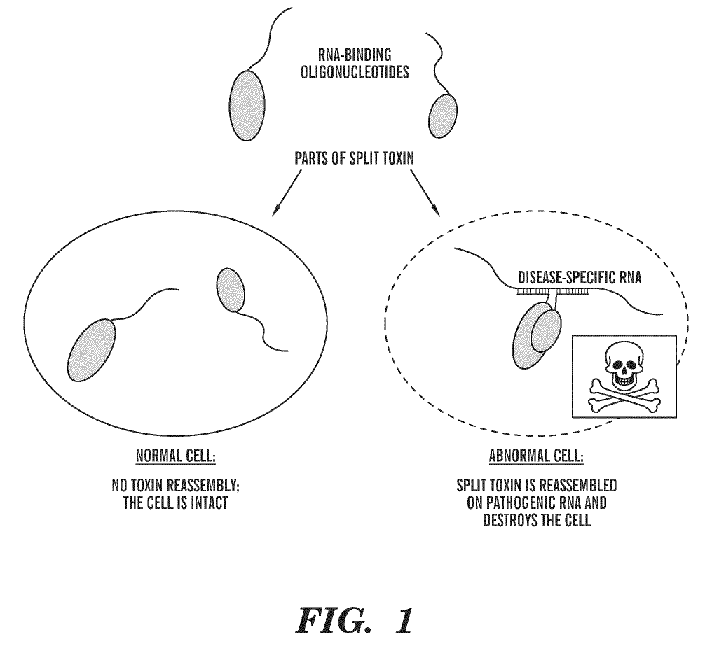

In the examples, we target RNA for TEL-AML1, which serves as a scaffold for ricin A reassembly (FIG. 1). Note that this RNA will be present only in the ALL cells as a result of the oncogenic chromosomal translocation. Still, there may be times in the cell cycle when ALL RNA is not expressed, thus allowing a small number of cancer cells to escape the killing action of a toxin. Repeated treatment may alleviate this problem. Another alternative is the reassembly of a toxin using the TEL-AML1 gene as a scaffold.

In the examples, we also focus on the RNA-based reassembly of a toxin considering the ease of the cytoplasmic delivery of a drug, as compared to its nuclear delivery, as well as the straightforwardness of RNA targeting by complementary oligonucleotides. But a gene-targeting scheme of this strategy is also likely, although it requires uncommon duplex DNA-invading oligomers (pseudocomplementary PNA5; Demidov et al., 2002), and special nuclear delivery vehicles will be needed in this case.

### Example 1

Genetically dissect the A chain of ricin into two fragments and clone them in bacteria as fusions with intein. The first part of experiments involves cloning two fragments of ricin A. Ricin A sequence is available as a clone in a pKK223-3 (a gift from Dr. Vitetta, Univ of Texas). Because it is only the A chain of ricin, without the B chain, there are no significant toxicities or risks from working with this plasmid or the protein product. From this plasmid, the inventors amplified ricin A fragments using PCR. The PCR products are cloned into the Twin vectors (NE Biolabs) as C-terminal fusions with intein. Split RTA genes are designed so that the corresponding protein fragments carry C- or N-terminal cysteines to facilitate their chemical attachment to oligonucleotides. Correct construction of the recombinant plasmids should be verified by sequencing to check that all necessary protein expression elements (promoter, initiation/stop codons and protein-coding genes) have right sequences, which are in correct frame with each other.

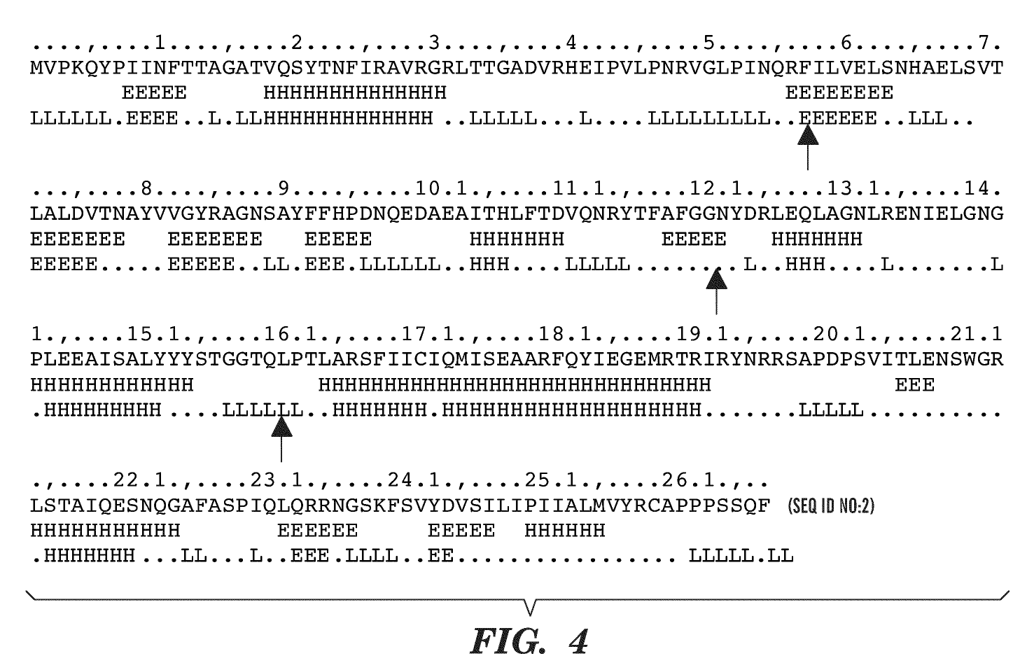

Since no data are available on dissection of the ricin A molecule, the inventors tested several variants of dissection in order to find a splitting site resulting in two complementing protein fragments that do not re-associate by themselves. Based on the 3D RTA structure (FIG. 2), one of the suggested sites for splitting will be at the interdomain I-II loop (central position 120-aa). The RTA secondary structure is shown schematically in FIG. 4. Based on this structure, two other splitting RTA points are feasible: position 52-aa in a large loop in the N-terminal part of the protein and at position 1 59-aa located in the helical C-terminal part of the protein. Both these alternative split points are located in unstructured regions, and splitting results in two fragments which make ⅓ and ⅔ of the protein. All splitting schemes will be tested for the lack of self-re-assembly and the background activity in the presence of attached ALL-specific oligonucleotides and control non-specific nucleic acids.

Optimal splitting of ricin A can be determined by assessing the structural conformation and assessing alternative splitting point and may require several cloning attempts, with the overall aim of achieving ricin A fragments that do not result in spontaneous reassembly, but is efficient at reassembly facilitated by complementary nucleic acid interactions. Additionally, it may require introduction of mutations to reduce ricin A self-assembly.

Plasmid pRTA coding for the full-length RTA (obtained from Univ. of Texas SW Medical Center) has been used as a PCR template for obtaining all six RTA gene fragments (FIG. 2); The RTA gene fragments thus obtained were inserted in the pTWIN vector as fusions with inteins, and the corresponding plasmids were first propagated in the E. coli XL10 cloning host cells, then transferred to the IPTG-inducible E. coli BL21-DE3 expression host cells.

### Example 2

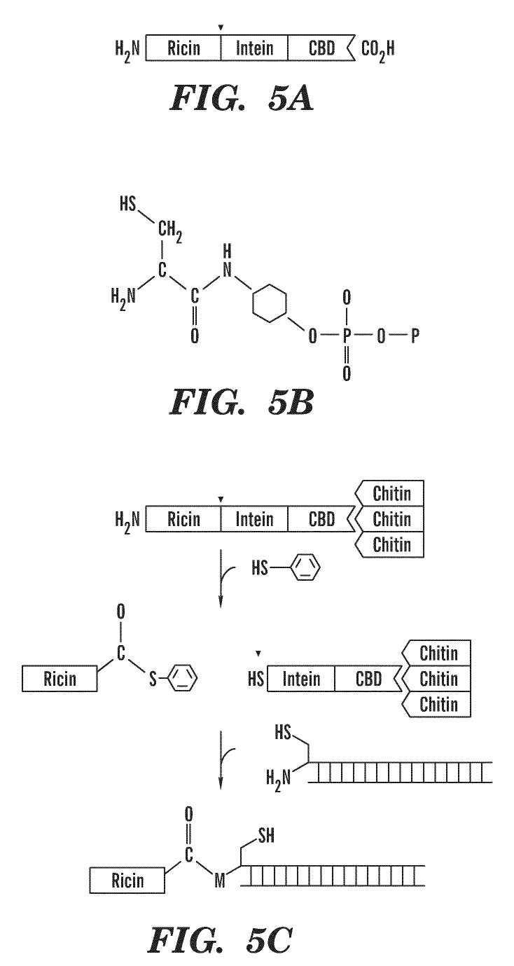

Attachment of probes (in this example the probes are oligonucleotides) to the RTA protein fragments via terminal cysteine, and to simultaneously split intein and to purify the protein-oligonucleotide conjugates. The inventors expressed the protein fusions in E. coli and isolated from soluble cellular fraction by loading onto the columns with chitin beads and by on-column splitting from intein. Splitting is performed in the presence of the oligonucleotides with pseudo-cysteine at the 5′ end (Burbulis et al., 2005). The scheme of intein-ricin chimera splitting in the presence of the modified oligonucleotide is shown in FIG. 5. This is an attractive chemistry for protein-oligonucleotide conjugation since it simultaneously allows one to achieve both protein purification and conjugation. Still, this is rather new conjugation approach tested by only a couple of groups, and it requires protein refolding, which could be problematic. Therefore, alternative conjugation chemistries can be performed.

Analysis of crude-cell protein preparations showed that IPTG induction of all E. coli BL21-DE3 clones transformed with the plasmids coding for different split RTA-intein fusions resulted in overexpression of the corresponding proteins, when induced at different temperatures, as shown in FIG. 2, showing an example for N2n and C2n expression.

However, some of the proteins were found to be overexpressed in the to insoluble fraction and form inactive inclusion bodies. In instances where the expressed split-effector protein fragment forms an inclusion bodies, the split-effector protein fragment can be expressed in cell-free systems and/or bacterial expression systems with E. coli strains which secrete the expressed protein into the culture medium as disclosed herein.

Where the split-effector protein fragment formed an inclusion bodies, such as those as shown in the insoluble fraction (FIG. 9), the inventors performed solubilization using urea solutions, in order to refold the split-effector protein fragments which were harvested from the insoluble fraction (as inclusion bodies) using drop-by-drop dilution method as previously employed herein for successful refolding of the split-effector protein fragments. Using this method, the inventors solubilized split EGPP-intein1 fusion protein fragments from inclusion bodies. The inventors also demonstrated this method of solubilization was effective with inclusion bodies of the N1n-RTA split-effector protein fragment and the subsequent isolation/purification on the chitin column, as shown in FIG. 11.

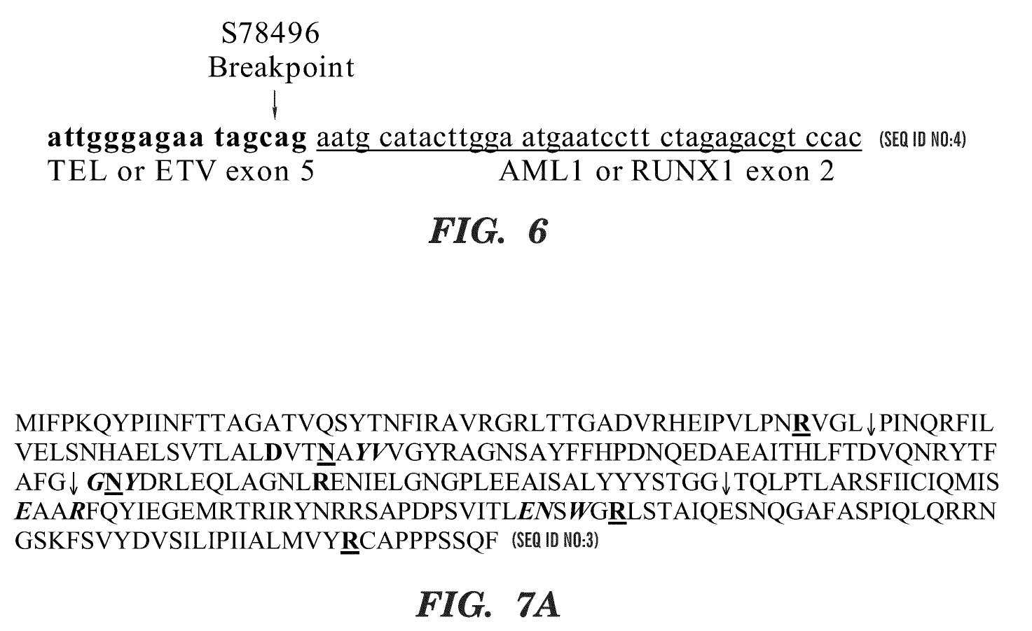

The target RNA site is case of major TEL-ALM1 of ALL disease is shown in FIG. 6. Two 15-20 nt long oligonucleotides are chosen from both sides from the breakpoint, and synthesized with 5′ pseudo-cystine modifications. This modification provides functional groups to link oligonucleotides to the protein fragments (Burbulis et al, 2005). The oligonucleotides can be purchased from any available source (eg Dalton Chem Lab Inc. Ontario, Canada). Note that possible individual differences in TEL-AML1 breakpoints and fusion sequences can be readily adjusted by choosing appropriate oligonucleotides.

The expression of split RTA-intein fusions may result in formation of inclusion bodies with the need for protein refolding. Protein refolding is a notoriously difficult technique; therefore optimization of correct folding the RTA fragments may be necessary.

### Example 3

In vitro functional activity of the split re-assembled ricin A. Ricin activity results in depurination of specific adenine in a model step-loop oligonucleotide or in the 28S rRNA. Depurinated oligos or RNA can be chemically split into two fragments, relative amounts of which can quantitatively measure ricin activity. Use of the ricin-splittable stem-loop RNA oligonucleotide and/or washed ribosomes as convenient substrates for testing the ricin nuclease activity (Argent et al, 2000; Garcia-Mayoral et al, 2005). Test samples consist of the toxin fragments with appended oligonucleotides bound to ALL-marker RNA. Intact RTA serves as a positive control, while the toxin fragments without the appended oligonucleiotides, and toxin fragments with appended oligonucleotides but without ALL-marker RNA serve as negative controls. Additional negative controls include all components of the complementing complex plus non-specific RNA. After the treatment with RTA-containing test and control samples, depurinated oligonucleotides or 28S rRNA are cleaved with aniline into two characteristic RNA fragments, which can be resolved by polyalcrylamide or agarose gel-electrophorosis (Argent et al, 2000).

If some of the negative controls display high background ricin activity, alternative variants of oligonucleotide attachment to the ricin fragments can be tested, in attempt to reduce the background as low as possible. As a first choice, oligonucleotides are attached to the C-terminus of the N-terminal fragment and to the N-terminus of the C-terminal fragment. If this results in high background, both oligonucleotides can be attached to the C-termini of both peptides. Based on our experience with reconstruction of split EGFP, the alternative scheme may result in a lower background of protein re-assembly.

These in vitro studies allow one to chose the optimal constructs (split point, RTA fragments arrangement, conjugation chemistry), as well as to verify the expected mechanism of action of split reconstituted RTA.

If the Biostability of oligonucleotides within the cells becomes an issue; modified nuclease-resistant oligonucleotides or PNA or pcPNA (Demidov et al, 1994) may be used (Cys-reactive SMCC PNA is commercially available).

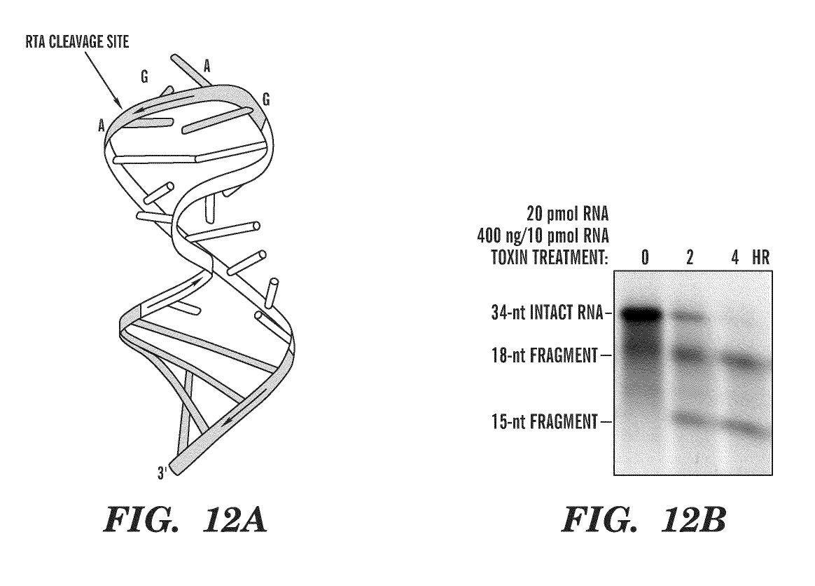

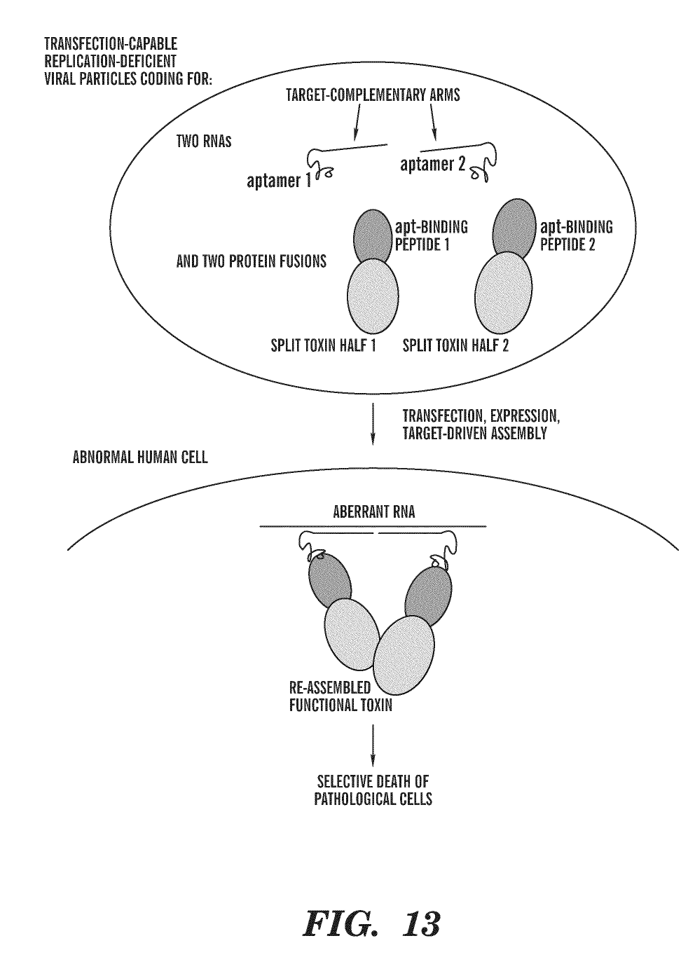

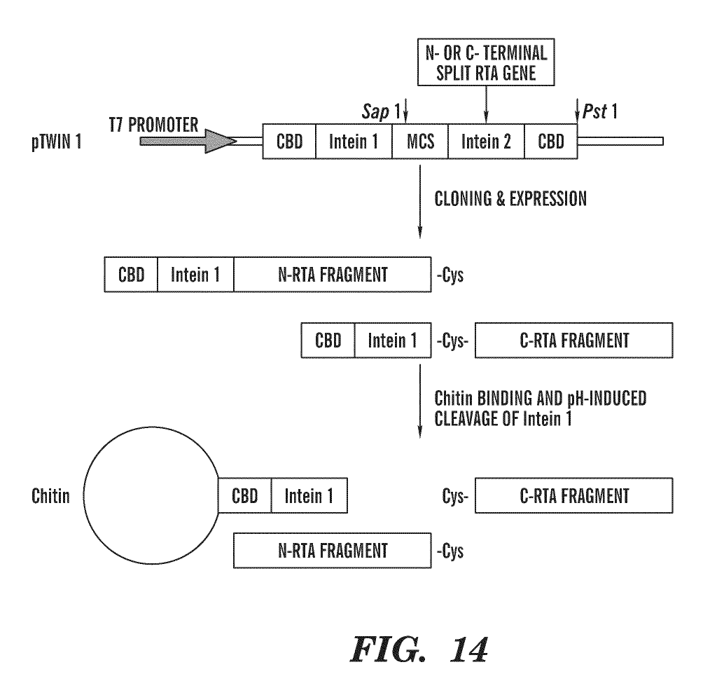

Based on the known RTA-splittable sequence within 28S rRNA, short 34-nt stem-loop RNA with the RTA target site was designed carrying dA instead of rA in the loop region for faster RTA-generated cleavage (such a replacement is known to significantly accelerate the RTA action). FIG. 12 shows that this RNA can be used for the gel electrophoresis-based fast testing of the RTA activity restoration: relative amounts of the two RNA fragments generated by the split-reassembled toxin treatment for a specific time will be a quantitative measure of ricin A activity in vitro, when compared to that of intact ricin A.

The split site the inventors selected for the splitting of the RTA results in no activity, i.e. no RNA-cleavage activity for the N-terminal N1n-RTA, because this split-effector protein fragment does not have the amino acids which comprise the RTA active site. In addition, no RTA activity, i.e. no RNA-cleavage activity occurs when N1n-RTA and C1n-RTA are simply mixed together, and thus indicates such RTA split-effector protein fragments require target mediated protein complementation for functional reassembly and the formation of an active effector RTA protein.

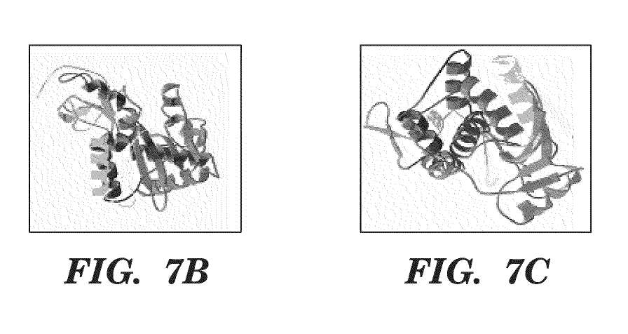

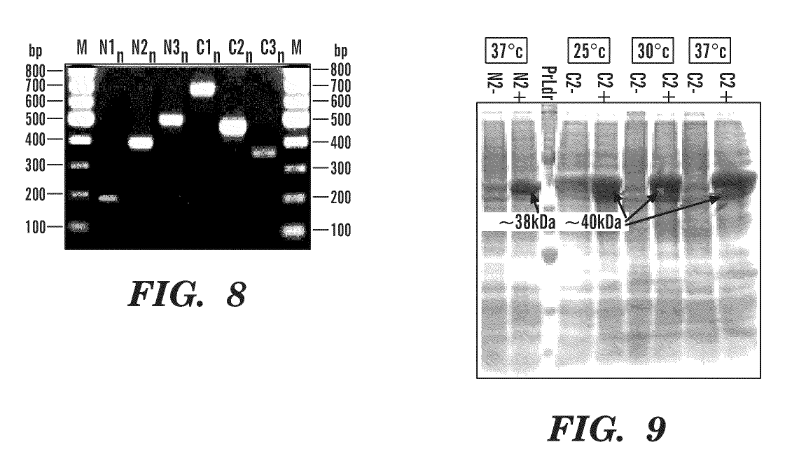

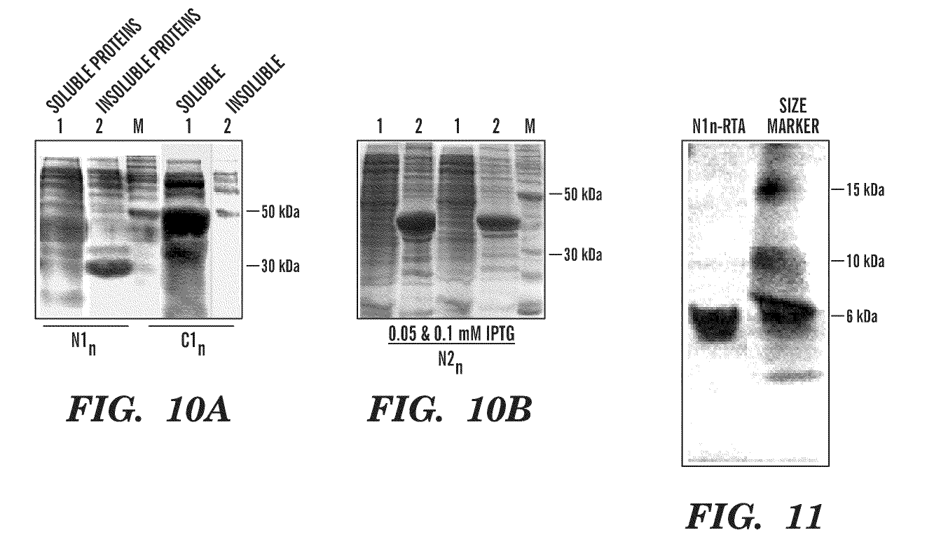

Using Cys-terminal peptide nucleic acid (PNA) oligomers as chemically and biologically stable nucleobase oligomers to be conjugated to the RTA half-proteins, the inventors assessed the suitability of the well-known Cu 2-Phenanthroline (Cu/Phe)-based protein coupling chemistry to conjugate the PNA oligomers. With Cu/Phe-treated Cys-PNAs, the inventors observed rapid quantitative dimerization of these oligomers without any degradation, whereas common oligonucleotides are degraded by Cu/Phe reagent. This result demonstrates that Cu/Phe coupling chemistry can be applied for Cys-PNA conjugation to the Cys-terminal split-RTA proteins. The inventors used this chemistry for conjugation of N1n-RTA with C1n-RTA, for assessment of the efficiency of reassembly of RTA split at the first point (FIG. 7). Alternatively, Cys-PNAs can be conjugated to terminally activated split-RTA proteins during the on-column intein cleavage using the MESNA-based chemistry and C-terminal protein fusions to intein2.

The inventors also developed an in vitro assay to identify functional reassembly of split-effector protein fragments, as demonstrated by analysis of some split RTA proteins using conjugation chemistry. This assay can also be utilized for functional reassembly of split-effector protein fragments conjugated to probes, for example nucleic acid probes such as oligonucleotide probes or polypeptide probes.

### Example 4

To functionality test on the cellular level, two complementing protein-oligonucleotide constructs can be injected into the target cells (eg ALL human cells). Healthy cells and placebo-injected ALL cells serve as controls. Estimation of survival rate provides a statistically significant number of cells and independent experiments. Specifically, two human ALL cell lines are used in the study; REH, a B-lineage ALL that contains the t(12;21) translocation and TEL/AML1 fusion (ATTC Cal No. CRL-8286), and the NALM6 B-lineage ALL that does not have the t(12;21) or the TEL/AML1 fusion (DSMZ Cat No. ACC 128). Additionally, primary ALL cells and the MTT assay as previously described (Holleman et al, 2004; Lugthart et al, 2005) are used to determine viability, with early and late apoptosis determined by FACS analysis. Optimal exposure time and concentration of the fusion-toxin can be tested in cellular assays using human ALL cell lines, and primary ALL cells with and without the TEL/AML1 fusion.

In vivo toxicity/efficacy can be assessed by using mice transplanted with bone marrow that is retrovirally transduced with the control vector or the vector containing the TEL-AML1 fusion gene (Fisher et al, 2005).

Following successful in vitro and in vivo assessment of the biomolecular conjugate, encapsulated formulations of split RTA-oligonucleotide conjugates can be developed as a candidate form of a new drug (drug-loaded polymeric nanoparticles or cationic liposomes as the drug-to-cytoplasm delivery vehicles). Following assessment of their stability in serum, they can then be tested in cells, and finally on animal models of the disease of interest, for example in ALL disease models.

A gene-targeting scheme of this strategy can also be developed as a future robust alternative. Based on this future development, a preventive approach to eradicate pre-cancer cells in newborns could be established.

The inventors analyzed the N1n-RTA/C1n-RTA split-effector protein fragment pair, including the in vitro and in vivo activity/reassembly testing of non-conjugated and conjugated proteins, especially in the presence of target RNA. The inventor also optimized protein expression for other split RTA proteins by varying the cell growth conditions to increase soluble expression and to develop the methods for refolding of split-RTA proteins from inclusion bodies.

### REFERENCES

The references cited herein and throughout the application are incorporated herein by reference.
1. Argent R H et al. (2000) Ribosome-mediated folding of partially unfolded ricin A-chain, J. Biol, Chem. 275(1 3):9263-9.
2. Bigalke H & Rummel A (2005) Medical aspects of toxin weapons. Toxicology 21 4(3):21 0-20.
3. Burbulis, I., Yamaguchi, K., Gordon, A., Carlson, R. & Brent, R. Using protein-DNA chimeras to detect and count small numbers of molecules. Nat. Methods. 2, 31-37 (2005).
4. Demidov, V. V. et al. (1994). Stability of peptide nucleic acids in human serum and cellular extracts. Biochem. Pharmacol. 48, 1310-1313.
5. Demidov, V. V. et al. (2002) Kinetics and mechanism of the DNA double helix invasion by pseudocomplementary peptide nucleic acids. Proc. Natl. Acad. Sci. USA 99, 5953-5958.
6. Demidov, V. V. et al. (2006) Fast complementation of split fluorescent protein triggered by DNA hybridization. Proc. Natl. Acad. Sci. USA 103, 2052-56.
7. Fischer M et al. (2005) Defining the oncogenic function of the TEL/AML1(ETV6/RUNX1) fusion protein in a mouse model. Oncogene, 24(51):7579-91.
8. Garcia-Mayoral F et al. (2005) Modeling the highly specific ribotoxin recognition of ribosomes. FEBS Lett, 579(30):6859-64.
9. Hartley M R & Lord J M (2004) Cytotoxic ribosome-inactivating lectins from plants. Biochim, Biophys. Acta 1701(1-2):1-14.
10. Holleman A, Cheok M H, den Boer M L, Yang W, Veerman A J P, Kazemier K M, Pei D, Cheng C, Pui C H, Relling M V, Janka-Schaub G E, Pieters R, Evans W E (2004) Gene expression patterns in drug resistance acute lymphoblastic leukemia and treatment response. New Engl. J. Med. 351:533-42.
11. Lord, J. M., Roberts, L. M, & Robertus, J. D. (1994) Ricin: structure, mode of action, and some current applications. FASEB J. 8, 201-208.
12. Lugthart 5, Cheok M H, den Boer M L, Yang W, Holleman A, Cheng C, Pui C H, Relling M V, Janka-Schaub G E, Pieters R, Evans W E (2005) Identification of genes associated with chemotherapy cross-resistance and treatment response in childhood acute lymphoblastic leukemia. Cancer Cell 7:375-86.
13. Mantis N J et al, (2006) Immunoglobulin A antibodies against ricin A and B subunits protect epithelial cells from ricin intoxication. Infect, Immun. 74(6):3455-62.
14. Michnick, S. W (2003) Protein fragment complementation strategies for biochemical network mapping. Curr Opin Biotechnol. 14, 610-617.
15. Mlsna D et al, (1993) Structure of recombinant ricin A chain at 2.3 A. Protein Sci. 2, 429-35
16. Pui, C. H., Rolling, M V. & Downing, J. R. Acute lymphoblastic leukemia. N Engl J. Med. 350, 1535-1548 (2004).
17. Pattrick, N. G, Richardson S C, Casolaro M, Ferruti P & Duncan R (2001) Poly(amidoamine)-mediated intracytoplasmic delivery of ricin A-chain and gelonin. J. Control Release 77(3)225-32.
18. Richardson, S. C., Pattrick, N. G, Man Y K, Ferruti P & Duncan R (2001) Poly(amidoamine)s as potential non-viral vectors: ability to form interpolyelectrolyte complexes and to mediate transfection in vitro. Biomacromolecules 2(3):1023-8.
19. Sachdeva M S (1998) Drug targeting systems for cancer chemotherapy. Expert Opin. Investig. Drugs. 7(11):1849-64.
20. Valencia-Burton M et at (2006). RNA visualization in live bacterial cells using fluorescent protein complementation (MS in preparation).
21. van Dongen J J et al (1999) Standardized RT-PCR analysis of fusion gene transcripts from chromosome aberrations in acute leukemia for detection of minimal residual disease. Leukemia 13(12)1901-28.
22. Wang S et al. (2006) A novel designed single domain antibody on 3-b structure of ricin A chain remarkably blocked ricin-induced cytotoxicity. Mol Immunol 43(11):191•2-9
23. Weston S A et al. (1994) X-ray structure of recombinant ricin A-chain at 1.8 A resolution.

J. Mol. Biol. 244(4):41 0-22.
24. Roche Applied Science. RTS Application Manual for Cell-Free Protein Expression; Roche Diagnostics GmbH: Penzberg (Germany), 2003 (https://www.roche-applied-science.com/sis/proteinexpression/index.jsp).
25. Novagen: Enhancing solubility during protein expression in E. coli; (http://www.emdbiosciences.com/html/NVG/solubility.html)
26. R N Rahman, T C Leow, M Basri, and A B Salleh (2005) Secretory expression of thermostable T1 lipase through bacteriocin release protein. Protein Expr. Purif. 40(2): 411-6

1.-157. (canceled)

158. A split biomolecular conjugate, comprising a split-effector molecule, wherein the split-effector polypeptide fragments are conjugated to one of at least two probes specific for a target nucleic acid or target polypeptide, wherein the target nucleic acid or target polypeptide is present in a cell suffering from a disease, malignancy or disorder, wherein binding of the probes to the target nucleic acid or polypeptide reconstitutes the effector molecule, and wherein the effector molecule is; lethal to the cell; and/or sensitizes the cell to another compound; and/or alleviates the disease, malignancy or disorder.

159. The split biomolecular conjugate of claim 158, wherein the split-effector molecule comprises at least two polypeptide fragments of an effector molecule; wherein the fragments; (a) are in an activated conformation; (b) are not active by themselves; (c) further comprise a probe; and (d) complement to reconstitute the active effector molecule in real time in the presence of a target nucleic acid or polypeptide.

160. A method for the treating or reducing the effects of a disease or disorder in a subject comprising;
a. administering to the subject an effective amount of a pharmaceutical composition of the split biomolecular conjugate of claim 158; comprising a split-effector molecule, wherein each of the split-effector polypeptide fragments are conjugated to at least one of two probes specific for a particular target nucleic acid or target polypeptide that is associated with a disease or disorder; and
b. formation of an active effector molecule, wherein the formation of an active effector molecule is facilitated by binding of at least two probes with the target nucleic acid or target polypeptide that is associated with a disease or disorder.

161. The method of claim 160, wherein the split effector molecule is a toxin molecule or fragment thereof.

162. The method of claim 161, wherein the toxin molecule is an immunotoxin or fragment thereof.

163. The method of claim 162, wherein the immunotoxin is a protein toxin.

164. The method of claim 163, wherein the protein toxin is a bacterial toxin or a plant toxin.

165. The method of claim 161, wherein the toxin molecule is a cytotoxic molecule.

166. The method of claim 160, wherein the effector molecule is a nuclease or has endonucleolytic activity.

167. The method of claim 160, wherein the split effector molecule is a proteolytic enzyme.

168. The method of claim 160, wherein the split effector molecule is capable of inducing a cell death pathway in the cell.

169. The method of claim 160, wherein the split effector molecule is a pro-apoptotic molecule.

170. The method of claim 160, wherein the split effector molecule is capable of inhibiting a cell death pathway or inducing a cell survival pathway in the cell.

171. The method of claim 160, wherein the effector molecule is an anti-apoptotic molecule.

172. The method of claim 160, wherein the effector molecule is a molecule or polypeptide that sensitizes the cell to one or more secondary agents.

173. The method of claim 160, wherein the effector molecule is a molecule that tags the target polypeptide for protein degradation.

174. The method of claim 160, wherein the disease or disorder due to a pathology causing nucleic acid.

175. The method of claim 160, wherein the disease or disorder is selected from a group comprising; cancer; neurological disease; degenerative disease; an inflammatory disease; a pathogen infection.

176. The method of claim 160, wherein the split-biomolecular conjugate is administered to the cell on preloaded polymetric nanoparticles and/or cataionic liposomes.

177. The conjugate of claim 158, wherein the split-effector molecule conjugated to the nucleic acid binding motif is expressed from an expression vector in said cell.

178. The method of claim 160, wherein the target nucleic acid comprises the pathology causing target nucleic acid sequence.

179. The method of claim 160, wherein the target nucleic acid is DNA.

180. The method of claim 160, wherein the target nucleic acid is RNA.

181. The conjugate of claim 158, wherein the target polypeptide comprises a pathogenic polypeptide.

182. The conjugate of claim 158, wherein the cell is in vitro and in vivo.

183. The method of claim 160, wherein the probe is a nucleic acid binding motif.

184. The method of claim 160, wherein the probe is a polypeptide detector protein.

185. The conjugate of claim 158, wherein the split-effector polypeptide fragments combine to form an active effector molecule in the presence of a particular target nucleic acid or target polypeptide that is capable of initiating a cell death pathway in the cell.

186. The conjugate of claim 158, wherein the split-effector polypeptide fragments combine to form an active effector molecule in the presence of a particular target nucleic acid or target polypeptide that is capable of degrading or inducing the degradation of the target nucleic acid or target polypeptide in the cell.

187. The conjugate of claim 158, wherein the split-effector polypeptide fragments combine to form an active effector molecule in the presence of a particular target nucleic acid or target polypeptide that is capable of sensitizing the cell to other secondary agents.

188. The conjugate of claim 158, wherein the split-effector polypeptide fragments combine to form an active effector molecule in the presence of a particular target nucleic acid or target polypeptide that is capable of initiating a cell survival pathway or inhibiting cell death in the cell.

189. The conjugate of claim 158, wherein the split-effector polypeptide fragments combine to form an active effector molecule in the presence of a particular target nucleic acid or target polypeptide that is capable of replacing a dysfunctional or lost polypeptide in the cell.

190. A method to measure the level of a pathogenic target nucleic acid or pathogenic polypeptide in a subject comprising;
a. administering to the subject an effective amount of a pharmaceutical composition of the split biomolecular conjugate comprising a split-detector molecule, wherein each of the split-detector polypeptide fragments are conjugated to at least one of two probes specific for a particular target nucleic acid or target polypeptide that is associated with a disease or disorder;
b. formation of an active detector molecule, wherein the formation of an active effector molecule is facilitated by binding of at least two probes with the target nucleic acid or target polypeptide that is associated with a disease or disorder;
c. measuring the level of the active detector molecule; and wherein the level of the active detector molecule is a measure of the target nucleic acid or pathogenic polypeptide in a subject.

191. The method of claim 190, wherein the detector polypeptide is selected from a group comprising; β-lactamase; DFHR; luciferase; fluorescent protein or variants or fragments thereof.

192. The method of claim 190, further comprising comparing the level of a pathogenic target nucleic acid or pathogenic polypeptide in a subject at a first timepoint with the level of a pathogenic target nucleic acid or pathogenic polypeptide at a second time point.


SEQUENCE LISTING (4 sequences)

SEQ ID NO: 1 - RNA, 34 nt, Artificial Sequence
  Description of Artificial Sequence Synthetic oligonucleotide
ggaauccugcucaguacgagaggaaccgcagguu

SEQ ID NO: 2 - protein, 267 aa, Ricinus communis
MVPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 3 - protein, 268 aa, Ricinus communis
MIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 4 - DNA, 54 bp, Homo sapiens
attgggagaatagcagaatgcatacttggaatgaatccttctagagacgtccac